SEQUENCE LISTING (3 sequences)

SEQ ID NO: 1 - DNA, 20 bp, Artificial
  PCR primer
tcccagagttcctccacaac

SEQ ID NO: 2 - DNA, 20 bp, Artificial
  PCR primer
attgagtgtccgctgcttct

SEQ ID NO: 3 - DNA, 20 bp, Artificial
  Fluorogenic probe
ctacgccacgctcatccgct

(19) United States
(12) Patent Application Publication
(10) Pub. No.: US 2006/0002932 A1
(43) Pub. Date: Jan. 5, 2006

(54) Methods and compositions for enhancement of immunity by in vivo depletion of immunosuppressive cell activity

(75) Inventor: Johannes W. Vieweg, Durham, NC (US)

Correspondence Address: MYERS BIGEL SIBLEY & SAJOVEC, PO BOX 37428, RALEIGH, NC, 27627, US

(73) Assignee: Duke University

(21) Appl. No.: 11/144,083

(22) Filed: Jun. 3, 2005

Related U.S. Application Data
(60) Provisional application No. 60/577,306, filed on Jun. 4, 2004.

(51) Int. Cl. A61K 39/395

(52) U.S. Cl. 424/144.1

(57) ABSTRACT

The present invention provides a method of enhancing an immune response in a subject, comprising administering to the subject a reagent that targets a cell having immunosuppressive activity, in an amount effective in reducing the immunosuppressive activity of the cell, thereby enhancing an immune response in the subject. Also provided are methods of treating cancer and infection in a subject by administering the reagents of this invention to a subject.

### STATEMENT OF PRIORITY

This application claims benefit, under 35 U.S.C. § 119(e), of U.S. Provisional Application No. 60/577,306, filed Jun. 4, 2004, the entire contents of which are incorporated herein by reference.

### STATEMENT OF GOVERNMENT SUPPORT

Studies described herein were supported in part by National Cancer Institute Grant No. 5R1CA098446. The Government has certain rights in this invention.

### BACKGROUND OF THE INVENTION

Active immunotherapy using vaccines or other immune enhancing approaches is receiving increasing interest in clinical oncology. Further enhancement of these approaches requires the disruption of regulatory pathways that suppress the activation of tumor-specific T-effector cells in the cancer patient. CD4⁺ T cell subsets that express the IL-2 receptor α-chain (CD25) have been shown to act in a regulatory capacity by suppressing the activation and function of other T-effector cells. The physiological role of these regulatory T cells is to protect the host against the development of autoimmunity by regulating immune responses against antigens expressed by normal tissues. Since tumor antigens are largely self-antigens, regulatory T cells may also prevent the tumor-bearing host from mounting an effective antitumor immune response. The present invention overcomes previous problems in the art by providing methods and compositions that improve and enhance the efficacy of vaccines and other immune based therapies in subjects by incorporating a strategy that reduces the numbers and/or activities of immunosuppressive cells. The resulting improved immune response will impact on clinical endpoints by reducing tumor burden and viral load and/or by enhancing disease free or overall survival.

### BRIEF DESCRIPTION OF THE DRAWINGS

FIGS. 1A-D show phenotypic and functional characteristics of human regulatory T cells.

FIGS. 2A-C show the selective elimination of regulatory T cells in vitro and enhancement of T cell responses following such depletion.

FIGS. 3A-D show the depletion of regulatory T cells in vivo.

FIG. 4 shows in vivo stimulation of tumor-specific T cell responses.

FIGS. 5A-C show cytokine secretion profiles in CD4⁺ T cells.

FIGS. 6A-D show the phenotypic and functional characteristics of human regulatory T cells.

FIGS. 7A-D show the selective elimination of regulatory T cells in vitro.

FIGS. 8A-D show the depletion of regulatory T cells in vivo.

FIGS. 9A-E show restoration of T_{reg} in patients' peripheral T cell pool (A); effects on the memory T cell pool (B); the frequency of interferon secreting T cells (C), the frequency of CD8⁺ responder T cells (D); and antigen-specific proliferation (E).

FIGS. 10A-C show in vivo stimulation of tumor-specific T cell responses.

### SUMMARY OF THE INVENTION

The present invention provides a method of enhancing an immune response in a subject, comprising administering to the subject a reagent that targets a cell having immunosuppressive activity, in an amount effective in reducing the immunosuppressive activity of the cell, thereby enhancing an immune response in the subject.

Also provided herein is a method of reducing the number of immunosuppressive cells in a subject in need thereof, comprising administering to the subject a reagent that targets a cell having immunosuppressive activity in an amount effective in reducing the number of immunosuppressive cells.

The present invention further provides a method of reducing the immunosuppressive effect of a cell in a subject, comprising administering to the subject an effective amount of a reagent that targets a cell having an immunosuppressive effect.

In a further embodiment, the present invention provides a method of treating cancer in a subject, comprising: a) administering to the subject a reagent that targets a cell having immunosuppressive activity in an amount effective in reducing the immunosuppressive activity of the cell; and b) administering to the subject a reagent that targets the cancer in the subject and/or elicits an immune response to the cancer cells of the subject.

Also provided herein is a method of treating an infection in a subject, comprising:
a) administering to the subject a reagent that targets a cell having immunosuppressive activity in an amount effective in reducing the immunosuppressive activity of the cell; and
b) administering to the subject a reagent that targets an infectious agent that is causing an infection in the subject.

### DETAILED DESCRIPTION OF THE INVENTION

As used herein, “a” or “an” or “the” can mean one or more than one. For example, “a” cell can mean one cell or a plurality of cells.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of this invention, dose, time, temperature, and the like, is meant to encompass variations of +20%, +10%, +5%, +1%, +0.5%, or even ±0.1% of the specified amount.

The present invention is based on the unexpected discovery that an immune response can be enhanced (e.g., in humans) in vivo by depletion and/or inactivation of immunosuppressive cells, such as regulatory T cells or immature myeloid suppressor cells. Thus, in one embodiment, the present invention provides a method of enhancing an immune response in a subject, comprising administering to the subject a reagent that targets a cell having immunosuppressive activity, in an amount effective in reducing the immunosuppressive activity of the cell, thereby enhancing an immune response in the subject.

Further provided herein is a method of reducing the number of immunosuppressive cells in a subject, comprising administering to the subject an effective amount of a reagent that targets a cell having immunosuppressive activity.

In addition, the present invention provides a method of reducing or eliminating the immunosuppressive effect or activity of a cell in a subject, comprising administering to the subject an effective amount of a reagent that targets a cell having an immunosuppressive effect or activity.

As indicated, a reduction in immunosuppressive effect or activity in a subject can be the result of a decrease in the number of immunosuppressive cells and/or the result of the elimination or reduction (e.g., suppression) of an activity or function of an immunosuppressive cell.

In further embodiments, the present invention provides a method of treating cancer in a subject, comprising: a) administering to the subject an effective amount of a reagent that targets a cell having immunosuppressive activity; and b) administering to the subject an effective amount of a reagent that targets the cancer in the subject and/or elicits an immune response to cancer cells of the subject.

The present invention further provides a method of treating or preventing an infection in a subject, comprising: a) administering to the subject an effective amount of a reagent that targets a cell having immunosuppressive activity; and b) administering to the subject a reagent that targets an infectious agent that is causing or contributing to an infection in the subject and/or can cause or contribute to an infection in the subject.

The reagent that targets a cell having immunosuppressive activity in a subject can be administered to the subject at any time relative to when the immunizing reagent is administered to the subject. Thus, in some embodiments of the treatment methods of this invention, the reagent that targets a cell having immunosuppressive activity can be administered to the subject prior to the immunization (e.g., hours, days, weeks before). In certain embodiments, the reagent can be administered to the subject with the proviso that it is not administered during the immunization (e.g., T cell priming) phase of the treatment. In other embodiments, the reagent can be administered during the immunization phase.

In the methods of this invention, the reagent that targets a cell having immunosuppressive activity can be, but is not limited to, an antibody, a ligand, an immunotoxin, a differentiation agent (e.g., all-trans retinoic acid) and/or a fusion protein that comprises a targeting moiety and a toxic moiety. By “target” or “targeting” is meant that the reagent specifically associates with (e.g., binds to or activates other reagents to bind to) the “target cell” (i.e., the cell having immunosuppressive activity) and exerts an effect on the target cell that reduces or eliminates the immunosuppressive activity of the target cell. The reduction and/or elimination of immunosuppressive activity (e.g., due to decreased number of cells and/or due to altered effect or activity of cells) can be determined according to methods described herein and as are well known in the art.

For example, an antibody or ligand of this invention can include, but is not limited to an antibody or ligand that binds CD25, an antibody or ligand that binds CTLA4, an antibody or ligand that binds GITR, an antibody or ligand that binds FOXP3, and an antibody or ligand that activates co-stimulatory molecules such as OX40 or CD40. Another example of an antibody or ligand of this invention is an antibody or ligand that binds a protein present on the surface of a CD25⁺ cell. One example of a ligand of this invention is interleukin-2 (IL-2) or a binding domain of IL-2 (e.g., a domain that binds to the IL-2 receptor on the surface of CD25⁺ cells). Other ligands of this invention include any ligands that bind a receptor present on the surface of CD25⁺ cells, as such ligands are known in the art and identified according to standard protocols.

When the cell to be targeted is an immature myeloid cell (e.g., HLA-DR⁻, CD33⁺), the reagent can be, for example, a CD33 depleting ligand or antibody.

In some embodiments, the reagent that associates with a target cell of this invention can be a fusion protein that comprises a first moiety that binds a target cell and a second moiety that imparts an effect on the target cell that reduces or eliminates immunosuppressive activity in a subject. An example of a fusion protein of this invention can be a fusion protein (e.g., chimeric protein) that comprises a catalytic domain of a toxin (e.g., diphtheria toxin) and also comprises a binding domain of a ligand that binds a receptor present on the surface of a target cell of this invention (e.g., a binding domain of interleukin-2 that binds to the interleukin-2 receptor present on the surface of CD25⁺ cells). In a particular embodiment, the fusion protein can be ONTAK™ (Ligand Pharmaceuticals).

In various embodiments of this invention, a reagent that results in reduction or elimination of immunosuppressive activity can be coupled (e.g., covalently bonded) to a suitable antibody or ligand either directly or indirectly (e.g., via a linker group). A direct reaction between a reagent and an antibody or ligand is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

Alternatively, a reagent and an antibody or ligand can be coupled via a linker group. A linker group can function as a spacer to distance an antibody or ligand from a reagent in order to avoid interference with binding capabilities. A linker group can also serve to increase the chemical reactivity of a substituent on a reagent or an antibody or ligand, and thus increase the coupling efficiency. An increase in chemical reactivity can also facilitate the use of reagents, or functional groups on reagents, which otherwise would not be possible.

A variety of bifunctional or polyfunctional reagents, both homo- and hetero-functional (such as those described in the catalog of the Pierce Chemical Co., Rockford, Ill.), as are known in the art, can be employed as the linker group. Coupling may be effected, for example, through amino groups, carboxyl groups, sulfhydryl groups or oxidized carbohydrate residues. There are numerous references describing such methodology, e.g., U.S. Pat. No. 4,671,958, the entire contents of which are incorporated by reference herein.

In cases where a reagent is more potent when free from the antibody or ligand of the present invention, it may be desirable to use a linker group that is cleavable during or upon internalization into a cell. A number of different cleavable linker groups have been described. The mechanisms for the intracellular release of a reagent from these linker groups include, for example, cleavage by reduction of a disulfide bond (e.g., U.S. Pat. No. 4,489,710), by irradiation of a photolabile bond (e.g., U.S. Pat. No. 4,625,014), by hydrolysis of derivatized amino acid side chains (e.g., U.S. Pat. No. 4,638,045), by serum complement-mediated hydrolysis (e.g., U.S. Pat. No. 4,671,958), and acid-catalyzed hydrolysis (e.g., U.S. Pat. No. 4,569,789).

In some embodiments, more than one reagent can be coupled to an antibody or ligand of this invention. In one embodiment, multiple molecules of a reagent are coupled to one antibody or ligand molecule. In another embodiment, more than one type of reagent can be coupled to one antibody or ligand. Immunoconjugates with more than one reagent can be prepared in a variety of ways, as known in the art. For example, more than one reagent can be coupled directly to an antibody or ligand molecule, or linkers that provide multiple sites for attachment can be used.

Alternatively, a carrier can be used. A carrier can bear the reagents in a variety of ways, including covalent bonding either directly or via a linker group. Suitable carriers include proteins, such as albumins (e.g., U.S. Pat. No. 4,507,234), as well as peptides and polysaccharides, such as aminodextran (e.g., U.S. Pat. No. 4,699,784). A carrier can also bear a reagent by noncovalent bonding or by encapsulation, such as within a liposome vesicle (e.g., U.S. Pat. Nos. 4,429,008 and 4,873,088). Carriers specific for radionuclide agents include radiohalogenated small molecules and chelating compounds (U.S. Pat. Nos. 4,735,792 and 4,673,562).

An antibody of this invention can be a polyclonal antibody, a monoclonal antibody, a single chain antibody, a bifunctional antibody, a humanized antibody, etc. and the production and characterization of such antibodies is standard in the art. An “antibody” of this invention can be employed in a variety of forms that allow for interaction with an antigen. For example, a number of molecules are known in the art that comprise antigen-binding sites that are capable of exhibiting immunological binding properties of an antibody molecule. The proteolytic enzyme papain preferentially cleaves IgG molecules to yield several fragments, two of which (the “F(ab)” fragments) each comprise a covalent heterodimer that includes an intact antigen-binding site. The enzyme pepsin is able to cleave IgG molecules to provide several fragments, including the “F(ab′)₂” fragment which comprises both-antigen-binding sites. An “Fv” fragment can be produced by preferential proteolytic cleavage of an IgM, IgG or IgA immunoglobulin molecule. Fv fragments are also derived using recombinant techniques known in the art. The Fv fragment includes a non-covalent V_{H}::V_{L} heterodimer including an antigen-binding site that retains much of the antigen recognition and binding capabilities of the native antibody molecule.

A single chain Fv (“sFv”) polypeptide is a covalently linked V_{H}::V_{L} heterodimer that is expressed from a gene fusion including V_{H}- and V_{L}-encoding genes linked by a peptide-encoding linker. A number of methods have been described to discern chemical structures for converting the naturally aggregated—but chemically separated—light and heavy polypeptide chains from an antibody V region into an sFv molecule that will fold into a three dimensional structure substantially similar to the structure of an antigen-binding site (U.S. Pat. Nos. 5,091,513; 5,132,405; and 4,946,778).

Each of the above-described molecules includes a heavy chain and a light chain CDR set, respectively interposed between a heavy chain and a light chain FR set that provide support to the CDRs and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1,” “CDR2” and “CDR3,” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRs. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs that form an antigen-binding surface. There are conserved structural regions of FRs that influence the folded shape of the CDR loops into certain “canonical” structures—regardless of the precise CDR amino acid sequence. Further, certain FR residues are known to participate in non-covalent interdomain contacts which stabilize the interaction of the antibody heavy and light chains.

A number of “humanized” antibody molecules comprising an antigen-binding site derived from a non-human immunoglobulin have been described, including chimeric antibodies having rodent V regions and their associated CDRs fused to human constant domains, rodent CDRs grafted into a human supporting FR prior to fusion with an appropriate human antibody constant domain and rodent CDRs supported by recombinantly veneered rodent FRs. These “humanized” molecules are designed to minimize unwanted immunological response toward rodent antihuman antibody molecules, which limits the duration and effectiveness of therapeutic applications of those moieties in humans.

As noted above, in some embodiments of the invention, antibodies and/or ligands of the present invention can be coupled to one or more reagents of this invention to reduce or eliminate immunosuppressive activity. Suitable reagents in this regard include, but are not limited to, radionuclides, differentiation inducers, drugs, toxins, and derivatives thereof. Examples of radionuclides include ⁹⁰Y, ¹²³I, ¹²⁵I, ¹³¹I, ¹⁸⁶Re, ¹⁸⁸Re, ²¹¹At, and ²¹²Bi. Examples of drugs include methotrexate, and pyrimidine and purine analogs. Examples of differentiation inducers include retinoids, dihydroxyvitamin D₃, all-trans retinoic acid (ATRA), fenretinide, α-cis-retinoic acid, phorbol esters and butyric acid. Examples of toxins include lectins (e.g., ricin, abrin, viscumin, modecin), diphtheria toxin, cholera toxin, gelonin, Pseudomonas exotoxin, Shigella toxin, and pokeweed antiviral protein.

Lectins are proteins, commonly derived from plants, which bind to carbohydrates. Among other activities, some lectins are toxic. Some of the most cytotoxic substances known are protein toxins of bacterial and plant origin (Frankel et al., Ann. Rev. Med. 37:125-142 (1986)). These molecules bind the cell surface and inhibit cellular protein synthesis. In ricin and abrin, the binding and toxic functions are contained in two separate protein subunits, the A and B chains. The ricin B chain binds to the cell surface carbohydrates and promotes the uptake of the A chain into the cell. Once inside the cell, the ricin A chain inhibits protein synthesis by inactivating the 60S subunit of the eukaryotic ribosome (Endo et al., J. Biol. Chem. 262: 5908-5912 (1987)). Other plant-derived toxins, which are single chain ribosomal inhibitory proteins, include pokeweed antiviral protein, wheat germ protein, gelonin, dianthins, momorcharins, trichosanthin and many others (Strip et al., FEBS Lett. 195:1-8 (1986)). Diphtheria toxin and Pseudomonas exotoxin A are also single chain proteins and their binding and toxicity functions reside in separate domains of the same protein chain with full toxin activity requiring proteolytic cleavage between the two domains. Pseudomonas exotoxin A has the same catalytic activity as diphtheria toxin. Conjugation of toxins to protein such as antibodies and other ligands is well known in the art (Olsnes et al., Immunol. Today 10:291-295 (1989); Vitetta et al., Ann. Rev. Immunol. 3:197-212 (1985)).

Cytotoxic drugs that interfere with critical cellular processes including DNA, RNA, and protein synthesis, can also be conjugated to antibodies and ligands and used for in vivo therapy. Such drugs include, but are not limited to, daunorubicin, doxorubicin, methotrexate, cyclophosphamide and mitomycin C.

In yet other embodiments of the invention, photosensitizers can be coupled to the antibodies or ligands of the invention for delivery directly to a target cell.

The target cell of this invention can be any cell that has immunosuppressive activity (e.g., a cell that suppresses the generation and/or activation of effector T cells) and in some embodiments, the cell can be a regulatory T cell, which can be for example, a CD25⁺ cell. Other cells of this invention include, but are not limited to, cells that express CTLA4 on the surface, cells that express GITR and/or cells that express FOXP3. Additional examples of cells of this invention include granulocytes, macrophages and immature myeloid suppressor cells (ImC). A cell can be identified as having immunosuppressive activity according to methods set forth in the EXAMPLES section provided herein, as well as according to art-known protocols standard in the art. Any such cell identified according to these teachings to have immunosuppressive activity can be a targeted cell of this invention.

As used herein, “regulatory T cells” include cells defined by the presence of the cell surface markers CD4, CD25, FOXP3, GITR and CTLA4, as well as any other T cell and/or other cell that is known or later identified to impart an immunosuppressive effect in a subject.

As used herein, “immature myeloid cells” or “immature myeloid suppressor cells” include cells defined by the presence of surface markers CD33, CD11B, CD11C and MHC Class I and the absence of lineage markers and MHC Class II markers.

In the methods of this invention, in addition to the administration to a subject of a reagent that targets a cell having immunosuppressive activity, a reagent is also administered to the subject that targets a cancer or infectious agent and/or elicits an immune response in the subject. Thus, in certain embodiments, removal or suppression of an interfering immunosuppressive activity in a subject by administration of the first reagent allows for the second reagent to impart an enhanced activity in the subject in the treatment of a cancer or infection.

The second reagent can be administered in any vehicle or form that allows the second reagent to impart a therapeutic effect. For example, when the second reagent elicits a n immune response, it can be administered to the subject by any means whereby an antigen is presented to cells of the subject's immune system. A variety of such immunization vehicles and systems are known in the art, including, but not limited to, proteins and peptides, dendritic cells and other immune cells, viral vectors, recombinant virus particles, vaccines (live, attenuated, killed, subunit, recombinant, protein, nucleic acid, etc.), nucleic acid (RNA or DNA), expression cassettes, plasmids, particles, liposomes and other carriers, etc. The selection, production, evaluation and administration protocols of such vehicles and systems are known in the art. The second reagent can also be a drug, small molecule, or other therapeutic compound or agent that acts to treat a cancer or infection in the subject.

A subject of this invention can include any animal in which cancer and infection is to be treated and/or prevented. In many embodiments, the methods of this invention are directed to humans, but subjects can also include, for example, animals such as dogs, cats, horses and other domestic and commercially important animals

In the methods of this invention wherein a cancer is being treated, the cancer can be, but is not limited to, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, hematological cancer (e.g., leukemia, lymphoma), multiple myeloma, Waldenstroom's macroglobulinemia, and/or heavy chain disease.

In the methods of this invention wherein an infection is treated, the infection can be caused by any pathogenic agent. Some examples include, but are not limited to, viral pathogens (e.g., hepatitis type A, hepatitis type B, hepatitis type C, influenza, varicella, adenovirus, herpesvirus, rhinovirus, echovirus, rotavirus, lentivirus, retrovirus, respiratory syncytial virus (RSV), papilloma virus, papova virus, cytomegalovirus, coronavirus, arbovirus, hantavirus, coxsackie virus, mumps virus, measles virus, rubella virus, polio virus, human immunodeficiency virus type I (HIV-I), and human immunodeficiency virus type II (HIV-II); prokaryotic pathogens (e.g., mycobacteria, rickettsia, Mycoplasma spp., Neisseria spp. and Legionella spp., chlamydia; and protozoal pathogens (e.g., Leishmania spp. and Trypanosoma spp).

In the methods of this invention, the reagent that acts to reduce or eliminate immunosuppressive activity of a cell in a subject can be administered to the subject at least zero, one, two, three, four, five, six, seven, eight, nine or ten days before a reagent that acts to elicit an immune response (e.g., to treat cancer or an infection) is administered to the subject. In certain embodiments, the reagent is administered only once to the subject. In other embodiments, the reagent can be administered more than once to the subject, at any interval. In some embodiments, the reagent can be administered so that a specific amount of the reagent is maintained in the subject for a period of time and in other embodiments, the reagent is administered such that it is present in the subject only transiently.

When the reagent of this invention is a fusion protein, the amount of fusion protein administered can be in a range from about 5 μg/kg to about 25 μg/kg, including any value in between (e.g., 6, 7, 8, 9, 10, 11, 12, 13, 14, 15, 16, 17, 18, 19, 20, 21, 22, 23 or 24 μg/kg).

In some embodiments, the reagent of this invention that is administered to a subject to treat cancer can be a dendritic cell loaded with messenger RNA encoding a tumor antigen specific for the cancer of the subject. In this embodiment, the amount of dendritic cells administered can be in the range of from about 3×10⁷ cells to about 10×10⁷ cells, including any value in between these two values (e.g., 4, 5, 6, 7, 8, or 9×10⁷ cells). The preparation and administration of DCs loaded with mRNA encoding a tumor antigen for the cancer of a subject of this invention is carried out according to protocols known in the art (e.g., U.S. Pat. Nos. 5,831,068; 5,853,719; 6,306,388; 6,387,701 and 6,670,186, the entire contents of each of which are incorporated by reference herein).

The present invention can be used to supplement any immune-based therapy, which can include, but is not limited to, active immunotherapy approaches (e.g., cancer vaccines, nucleic acid vaccines, ganglioside vaccines, heat shock proteins, etc., as well as any other agent that stimulates T cells); passive immunotherapy (e.g., adoptive transfer of T-cells or other immune cells), and “classical adjuvants” (e.g., proteins, peptides, oligonucleotides, si RNAs, recombinatorial therapeutics, etc.) that have immune-enhancing effects.

Methods for detecting an immune response can include, but are not limited to, antibody detection assays such as, for example, EIA (enzyme immunoassay); ELISA (enzyme linked immunosorbent assay); agglutination reactions; precipitation/flocculation reactions, immunoblots (Western blot; dot/slot blot); (RIA) radioimmunoassays; immunodiffusion assays; histochemical assays; immunofluorescence assays (FACS); chemiluminescence assays, library screens, expression arrays, etc. Assays for the detection of T cell responses include, but are not limited to, delayed-type hypersensitivity responses; in vitro T cell proliferation responses (e.g., measured by incorporation of radioactive nucleotides), library screens, expression arrays, T cell cytokine responses (e.g., measured by ELISA or other related immuno-assays or RT-PCR for specific cytokine mRNA), as well as any other assay known for measuring a B cell and/or T cell immune response in a subject.

Also as used herein, the term “fusion protein or chimeric protein” includes a protein or polypeptide comprising a first amino acid sequence that can be a peptide, a fragment of a protein or a whole protein that is linked or joined to a second amino acid sequence that can be a peptide, a fragment of a protein or a whole protein and wherein the first and second amino acid sequences are not linked or joined in the same way in nature.

Also as used herein, the terms peptide and polypeptide are used to describe a chain of amino acids, which correspond to those encoded by a nucleic acid. A peptide usually describes a chain of amino acids of from two to about 30 amino acids and polypeptide usually describes a chain of amino acids having more than about 30 amino acids. The term polypeptide can refer to a linear chain of amino acids or it can refer to a chain of amino acids, which have been processed and folded into a functional protein. It is understood, however, that 30 is an arbitrary number with regard to distinguishing peptides and polypeptides and the terms may be used interchangeably for a chain of amino acids around 30. The peptides and polypeptides of the present invention are obtained by isolation and purification of the peptides and polypeptides from cells where they are produced naturally or by expression of a recombinant and/or synthetic nucleic acid encoding the peptide or polypeptide. The peptides and polypeptides of this invention can be obtained by chemical synthesis, by proteolytic cleavage of a polypeptide and/or by synthesis from nucleic acid encoding the peptide or polypeptide.

It is also understood that the peptides and polypeptides of this invention may also contain conservative substitutions where a naturally occurring amino acid is replaced by one having similar properties and which does not alter the function of the polypeptide. Such conservative substitutions are well known in the art. Thus, it is understood that, where desired, modifications and changes, which are distinct from the substitutions which enhance immunogenicity, may be made in the nucleic acid and/or amino acid sequence of the peptides and polypeptides of the present invention and still obtain a peptide or polypeptide having like or otherwise desirable characteristics. Such changes may occur in natural isolates or may be synthetically introduced using site-specific mutagenesis, the procedures for which, such as mis-match polymerase chain reaction (PCR), are well known in the art. One of skill in the art will also understand that polypeptides and nucleic acids that contain modified amino acids and nucleotides, respectively (e.g., to increase the half-life and/or the therapeutic efficacy of the molecule), can be used in the methods of the invention.

An antigen of this invention can be a whole protein, a fragment of a protein, a synthetic antigen, an immunogenic peptide, an antibody and/or T cell epitope and/or a T cell stimulatory peptide. Identification and/or production of immunogenic peptides, T cell stimulatory peptides, antibody and T cell epitopes and the like is carried out by methods well known in the art.

For example, an antigen of this invention can include, but is not limited to, influenza antigens, polio antigens, tetanus toxin and other tetanus antigens, herpes antigens [e.g., CMV, EBV, HSV, VZV (chicken pox virus)], mumps antigens, measles antigens, rubella antigens, diphtheria toxin or other diphtheria antigens, pertussis antigens, hepatitis (e.g., hepatitis A, hepatitis B and hepatitis C) antigens, smallpox antigens, adenovirus antigens, HIV antigens, or any other vaccine antigen, as would be recognized in the art. An antigen of this invention can also be a “custom antigen” specific for that subject.

A cancer antigen (i.e., an antigen specifically associated with cancer cells) of this invention can include, for example, HER2/neu and BRCA1 antigens for breast cancer, MART-1/MelanA, gp100, tyrosinase, TRP-1, TRP-2, NY-ESO-1, CDK-4, β-catenin, MUM-1, Caspase-8, KIAA0205, HPVE7, SART-1, PRAME, and p15 antigens, members of the MAGE family, the BAGE family (such as BAGE-1), the DAGE/PRAME family (such as DAGE-1), the GAGE family, the RAGE family (such as RAGE-I), the SMAGE family, NAG, TAG-72, CA125, mutated proto-oncogenes such as p21ras, mutated tumor suppressor genes such as p53, tumor associated viral antigens (e.g., HPV16 E7), the SSX family, HOM-MEL-55, NY-COL-2, HOM-HD-397, HOM-RCC-1.14, HOM-HD-21, HOM-NSCLC-11, HOM-MEL-2.4, HOM-TES-11, RCC-3.1.3, NY-ESO-1, and the SCP family. Members of the MAGE family include, but are not limited to, MAGE-1, MAGE-2, MAGE-3, MAGE-4 and MAGE-11. Members of the GAGE family include, but are not limited to, GAGE-I, GAGE-6. See, e.g., review by Van den Eynde and van der Bruggen (1997) in Curr. Opin. Immunol. 9: 684-693, Sahin et al. (1997) in Curr. Opin. Immunol. 9: 709-716, and Shawler et al. (1997), the entire contents of which are incorporated by reference herein for their teachings of cancer antigens.

The cancer antigen can also be, but is not limited to, human epithelial cell mucin (Muc-1; a 20 amino acid core repeat for Muc-1 glycoprotein, present on breast cancer cells and pancreatic cancer cells), MUC-2, MUC-3, MUC-18, the Ha-ras oncogene product, carcino-embryonic antigen (CEA), the raf oncogene product, CA-125, GD2, GD3, GM2, TF, sTn, gp75, EBV-LMP 1 & 2, HPV-F4, 6, 7, prostatic serum antigen (PSA), prostate-specific membrane antigen (PSMA), alpha-fetoprotein (AFP), CO17-1A, GA733, gp72, p53, the ras oncogene product, p-HCG, gp43, HSP-70, p17 mel, HSP-70, gp43, HMW, HOJ-1, melanoma gangliosides, TAG-72, mutated proto-oncogenes such as p21ras, mutated tumor suppressor genes such as p53, estrogen receptor, milk fat globulin, telomerases, nuclear matrix proteins, prostatic acid phosphatase, protein MZ2-E, polymorphic epithelial mucin (PEM), folate-binding-protein LK26, truncated epidermal growth factor receptor (EGFR), Thomsen-Friedenreich (T) antigen, GM-2 and GD-2 gangliosides, polymorphic epithelial mucin, folate-binding protein LK26, human chorionic gonadotropin (HCG), pancreatic oncofetal antigen, cancer antigens 15-3,19-9, 549, 195, squamous cell carcinoma antigen (SCCA), ovarian cancer antigen (OCA), pancreas cancer associated antigen (PaA), mutant K-ras proteins, mutant p53, and chimeric protein p210_{BCR-ABL} and tumor associated viral antigens (e.g., HPV16 E7).

The cancer antigen of this invention can also be an antibody produced by a B cell tumor (e.g., B cell lymphoma; B cell leukemia; myeloma; hairy cell leukemia), a fragment of such an antibody, which contains an epitope of the idiotype of the antibody, a malignant B cell antigen receptor, a malignant B cell immunoglobulin idiotype, a variable region of an immunoglobulin, a hypervariable region or complementarity determining region (CDR) of a variable region of an immunoglobulin, a malignant T cell receptor (TCR), a variable region of a TCR and/or a hypervariable region of a TCR. In one embodiment, the cancer antigen of this invention can be a single chain antibody (scFv), comprising linked V_{H}, and V_{L} domains, which retains the conformation and specific binding activity of the native idiotype of the antibody.

A cancer antigen of this invention can also be an antigen specific for a tumor present in a particular subject (e.g., an autologous tumor antigen).

The present invention is in no way limited to the cancer antigens listed herein. Other cancer antigens be identified, isolated and cloned by methods known in the art such as those disclosed in U.S. Pat. No. 4,514,506, the entire contents of which are incorporated by reference herein.

The cancer to be treated by administration to a subject of a reagent of this invention can be, but is not limited to, B cell lymphoma, T cell lymphoma, myeloma, leukemia, chronic myeloid leukemia, acute myeloid leukemia, chronic lymphocytic leukemia, acute lymphocytic leukemia, hematopoietic neoplasias, thymoma, lymphoma, sarcoma, lung cancer, liver cancer, non-Hodgkins lymphoma, Hodgkins lymphoma, uterine cancer, adenocarcinoma, breast cancer, pancreatic cancer, colon cancer, lung cancer, renal cancer, bladder cancer, liver cancer, prostate cancer, ovarian cancer, primary or metastatic melanoma, squamous cell carcinoma, basal cell carcinoma, brain cancer, angiosarcoma, hemangiosarcoma, head and neck carcinoma, thyroid carcinoma, soft tissue sarcoma, bone sarcoma, testicular cancer, uterine cancer, cervical cancer, gastrointestinal cancer, and any other cancer now known or later identified (see, e.g., Rosenberg (1996) Ann. Rev. Med. 47:481-491, the entire contents of which are incorporated by reference herein).

Infectious agent antigens of this invention can include, but are not limited to, antigenic peptides or proteins encoded by the genomes of Hepadnaviridae including hepatitis A, B, C, D, E, F, G, etc. (e.g., HBsAg, HBcAg, HBeAg); Flaviviridae including human hepatitis C virus (HCV), yellow fever virus and dengue viruses; Retroviridae including human immunodeficiency viruses (HIV) (e.g., gp120, gp160, gp41, an active (i.e., antigenic) fragment of gp120, an active (i.e., antigenic) fragment of gp160 and/or an active (i.e., antigenic) fragment of gp41) and human T lymphotropic viruses (HTLV1 and HTLV2); Herpesviridae including herpes simplex viruses (HSV-1 and HSV-2), Epstein Barr virus (EBV), cytomegalovirus, varicella-zoster virus (VZV), human herpes virus 6 (HHV-6) human herpes virus 8 (HHV-8), and herpes B virus; Papovaviridae including human papilloma viruses; Rhabdoviridae including rabies virus; Paramyxoviridae including respiratory syncytial virus; Reoviridae including rotaviruses; Bunyaviridae including hantaviruses; Filoviridae including Ebola virus; Adenoviridae; Parvoviridae including parvovirus B-19; Arenaviridae including Lassa virus; Orthomyxoviridae including influenza viruses (e.g., NP, HA antigen); Poxyiridae including Orf virus, molluscum contageosum virus, smallpox virus and Monkey pox virus; Togaviridae including Venezuelan equine encephalitis virus; Coronaviridae including corona viruses such as the severe acute respiratory syndrome (SARS) virus; and Picornaviridae including polioviruses; rhinoviruses; orbiviruses; picodnaviruses; encephalomyocarditis virus (EMV); Parainfluenza viruses, adenoviruses, Coxsackieviruses, Echoviruses, Rubeola virus, Rubella virus, human papillomaviruses, Canine distemper virus, Canine contagious hepatitis virus, Feline calicivirus, Feline rhinotracheitis virus, TGE virus (swine), Foot and mouth disease virus, simian virus 5, human parainfluenza virus type 2, human metapneuomovirus, enteroviruses, and any other pathogenic virus now known or later identified (see, e.g., Fundamental Virology, Fields et al., Eds., 3^{rd} ed., Lippincott-Raven, New York, 1996, the entire contents of which are incorporated by reference herein for the teachings of pathogenic viruses).

The antigen of this invention can be an antigenic peptide or protein of a pathogenic microorganism, which can include but is not limited to, Rickettsia, Chlamydia, Mycobacteria, Clostridia, Corynebacteria, Mycoplasma, Ureaplasma, Legionella, Shigella, Salmonella, pathogenic Escherichia coli species, Bordatella, Neisseria, Treponema, Bacillus, Haemophilus, Moraxella, Vibrio, Staphylococcus spp., Streptococcus spp., Campylobacter spp., Borrelia spp., Leptospira spp., Erlichia spp., Klebsiella spp., Pseudomonas spp., Helicobacter spp., and any other pathogenic microorganism now known or later identified (see, e.g., Microbiology, Davis et al, Eds., 4^{th} ed., Lippincott, New York, 1990, the entire contents of which are incorporated herein by reference for the teachings of pathogenic microorganisms).

Antigens of this invention can be antigenic peptides or proteins from pathogenic protozoa, including, but not limited to, Plasmodium species (e.g., malaria antigens), Babeosis species, Schistosoma species, Trypanosoma species, Pneumocystis camii, Toxoplasma species, Leishmania species, and any other protozoan pathogen now known or later identified.

Antigens of this invention can also be antigenic peptides or proteins from pathogenic yeast and fungi, including, but not limited to, Aspergillus species, Candida species, Cryptococcus species, Histoplasma species, Coccidioides species, and any other pathogenic fungus now known or later identified.

Transplantation antigens for use as an antigen of this invention include, but are not limited to, different antigenic specificities of HLA-A, B and C Class I proteins. Different antigenic specificities of HLA-DR, HLA-DQ, HLA-DP and HLA-DW Class II proteins can also be used (WHO Nomenclature Committee, Immunogenetics 16:135 (1992); Hensen et al., in Fundamental Immunology, Paul, Ed., pp. 577-628, Raven Press, New York, 1993; NIH Genbank and EMBL data bases).

The present invention also contemplates the use of allergic antigens or allergens, which can include, but are not limited to, environmental allergens such as dust mite allergens; plant allergens such as pollen, including ragweed pollen; insect allergens such as bee and ant venom; and animal allergens such as cat dander, dog dander and animal saliva allergens.

The present invention also provides autoantigens as an antigen of this invention, for example, to enhance self-tolerance to an autoantigen in a subject. Exemplary autoantigens of this invention can include, but are not limited to, myelin basic protein, islet cell antigens, insulin, collagen and human collagen glycoprotein 39, muscle acetylcholine receptor and its separate polypeptide chains and peptide epitopes, glutamic acid decarboxylase and muscle-specific receptor tyrosine kinase.

As indicated, the present invention provides a reagent for immunization of a subject in whom immunosuppressive activity has been altered. Such a reagent can be a nucleic acid encoding a protein or peptide reagent of this invention. The nucleic acid can be administered to the subject and/or the nucleic acid can be expressed in vitro to produce the protein or peptide that is administered to the subject.

“Nucleic acid” as used herein refers to single- or double-stranded molecules which may be DNA, comprised of the nucleotide bases A, T, C and G, or RNA, comprised of the bases A, U (substitutes for T), C, and G. The nucleic acid may represent a coding strand or its complement. Nucleic acids may be identical in sequence to the sequence, which is naturally occurring or may include alternative codons, which encode the same amino acid as that which is found in the naturally occurring sequence. Furthermore, nucleic acids may include codons, which represent conservative substitutions of amino acids as are well known in the art. The nucleic acids of this invention can also comprise any nucleotide analogs and/or derivatives as are well known in the art.

As used herein, the term “isolated nucleic acid” means a nucleic acid separated or substantially free from at least some of the other components of the naturally occurring organism, for example, the cell structural components commonly found associated with nucleic acids in a cellular environment and/or other nucleic acids. The isolation of nucleic acids can therefore be accomplished by well-known techniques such as cell lysis followed by phenol plus chloroform extraction, followed by ethanol precipitation of the nucleic acids. The nucleic acids of this invention can be isolated from cells according to methods well known in the art for isolating nucleic acids. Alternatively, the nucleic acids of the present invention can be synthesized according to standard protocols well described in the literature for synthesizing nucleic acids. Modifications to the nucleic acids of the invention are also contemplated, provided that the essential structure and function of the peptide or polypeptide encoded by the nucleic acid are maintained.

The nucleic acid encoding the peptide or polypeptide of this invention can be part of a recombinant nucleic acid construct comprising any combination of restriction sites and/or functional elements as are well known in the art that facilitate molecular cloning and other recombinant DNA manipulations. Thus, the present invention further provides a recombinant nucleic acid construct comprising a nucleic acid encoding a peptide and/or polypeptide of this invention.

The present invention further provides a vector comprising a nucleic acid encoding a peptide and/or polypeptide of this invention. The vector can be an expression vector which contains all of the genetic components required for expression of the nucleic acid in cells into which the vector has been introduced, as are well known in the art. The expression vector can be a commercial expression vector or it can be constructed in the laboratory according to standard molecular biology protocols. The expression vector can comprise, for example, viral nucleic acid including, but not limited to, vaccinia virus, adenovirus, retrovirus, alphavirus and/or adeno-associated virus nucleic acid. The nucleic acid or vector of this invention can also be in a liposome or a delivery vehicle, which can be taken up by a cell via receptor-mediated or other type of endocytosis.

The nucleic acid of this invention can be in a cell, which can be a cell expressing the nucleic acid whereby a peptide and/or polypeptide of this invention is produced in the cell. In addition, the vector of this invention can be in a cell, which can be a cell expressing the nucleic acid of the vector whereby a peptide and/or polypeptide of this invention is produced in the cell. It is also contemplated that the nucleic acids and/or vectors of this invention can be present in a host (e.g., a bacterial cell, a cell line, a transgenic animal, etc.) that can express the peptides and/or polypeptides of the present invention.

For production of the polypeptides and/or peptides of this invention in prokaryotes, there are numerous E. coli (Escherichia coli) expression vectors known to one of ordinary skill in the art useful for the expression of nucleic acid encoding proteins such as fusion or chimeric proteins. Other microbial hosts suitable for use include bacilli, such as Bacillus subtilis, and other enterobacteria, such as Salmonella, Serratia, as well as various Pseudomonas species. These prokaryotic hosts can support expression vectors that will typically contain sequences compatible with the host cell (e.g., an origin of replication). In addition, any number of a variety of well-known promoters will be present, such as the lactose promoter system, a tryptophan (Trp) promoter system, a beta-lactamase promoter system, or a promoter system from phage lambda. The promoters will typically control expression, optionally with an operator sequence and have ribosome binding site sequences for example, for initiating and completing transcription and translation. If necessary, an amino terminal methionine can be provided by insertion of a Met codon 5′ and in-frame with the coding sequence of the protein. Also, the carboxy-terminal extension of the protein can be removed using standard oligonucleotide mutagenesis procedures.

Additionally, yeast expression systems and baculovirus systems, which are well known in the art, can be used to produce the chimeric peptides and polypeptides of this invention.

The vectors of this invention can be transferred into a cell by well-known methods, which vary depending on the type of cell host. For example, calcium chloride transfection is commonly utilized for prokaryotic cells, whereas calcium phosphate treatment, lipofection or electroporation can be used for other cell hosts.

The nucleic acid encoding the peptides and polypeptides of this invention can be any nucleic acid that functionally encodes the peptides and polypeptides of this invention. To functionally encode the peptides and polypeptides (i.e., allow the nucleic acids to be expressed), the nucleic acid of this invention can include, for example, antibiotic resistance markers, origins of replication and/or expression control sequences, such as, for example, a promoter (constitutive or inducible), an enhancer and necessary information processing sites, such as ribosome binding sites, RNA splice sites, polyadenylation sites and transcriptional terminator sequences.

Examples of expression control sequences useful in this invention include promoters derived from metallothionien genes, actin genes, immunoglobulin genes, CMV, SV40, adenovirus, bovine papilloma virus, etc. A nucleic acid encoding a selected peptide or polypeptide can readily be determined based upon the genetic code for the amino acid sequence of the selected peptide or polypeptide and many nucleic acids will encode any selected peptide or polypeptide. Modifications in the nucleic acid sequence encoding the peptide or polypeptide are also contemplated. Modifications that can be useful are modifications to the sequences controlling expression of the peptide or polypeptide to make production of the peptide or polypeptide inducible or repressible as controlled by the appropriate inducer or repressor. Such methods are standard in the art. The nucleic acid of this invention and its complementary sequence can be generated by means standard in the art, such as by recombinant nucleic acid techniques and by synthetic nucleic acid synthesis or in vitro enzymatic synthesis.

In certain embodiments of this invention, a reagent of this invention can be combined with an adjuvant. Thus, in some embodiments, the present invention provides a composition comprising a reagent of this invention and an adjuvant in the form of an amino acid sequence, as well as a nucleic acid encoding a reagent of this invention and a nucleic acid encoding an adjuvant. The adjuvant, in the form of an amino acid sequence, can be a component of the reagent and/or a separate component of the composition comprising the reagent of this invention. The adjuvant in the form of a nucleic acid, can be a component of the nucleic acid encoding the reagent and/or a separate component of the composition comprising the nucleic acid encoding the reagent of this invention. An adjuvant of this invention can be an amino acid sequence that is a peptide, a protein fragment or a whole protein that functions as the adjuvant, or the adjuvant can be a nucleic acid encoding a peptide, protein fragment or whole protein that functions as an adjuvant. An adjuvant can also be a small molecule or chemical compound that can be combined with a reagent of this invention. As used herein, “adjuvant” describes a substance, which can be any immunomodulating substance capable of being combined with the reagent of this invention to enhance, improve or otherwise modulate an immune response in a subject without deleterious effect on the subject.

An adjuvant of this invention can be, but is not limited to, for example, an immunostimulatory cytokine (including, but not limited to, GM/CSF, interleukin-2, interleukin-12, interferon-gamma, interleukin-4, tumor necrosis factor-alpha, interleukin-1, saponin, hematopoietic factor flt3L, CD40L, B7.1 co-stimulatory molecules and B7.2 co-stimulatory molecules), SYNTEX adjuvant formulation 1 (SAF-1) composed of 5 percent (wt/vol) squalene (DASF, Parsippany, N.J.), 2.5 percent Pluronic, L121 polymer (Aldrich Chemical, Milwaukee), and 0.2 percent polysorbate (Tween 80, Sigma) in phosphate-buffered saline. Suitable adjuvants also include an aluminum salt such as aluminum hydroxide gel (alum), aluminum phosphate, or algannmulin, but may also be a salt of calcium, iron or zinc, or may be an insoluble suspension of acylated tyrosine, or acylated sugars, cationically or anionically derivatized polysaccharides, or polyphosphazenes.

Other adjuvants are well known in the art and include QS-21, Freund's adjuvant (complete and incomplete), aluminum hydroxide, N-acetyl-muramyl-L-threonyl-D-isoglutamine (thr-MDP), N-acetyl-normuramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE) and RIBI, which contains three components extracted from bacteria, monophosphoryl lipid A, trealose dimycolate and cell wall skeleton (MPL+TDM+CWS) in 2% squalene/Tween 80 emulsion.

Additional adjuvants can include, for example, a combination of monophosphoryl lipid A, preferably 3-de-O-acylated monophosphoryl. lipid A (3D-MPL) together with an aluminum salt. An enhanced adjuvant system involves the combination of a monophosphoryl lipid A and a saponin derivative, particularly the combination of QS21 and 3D-MPL as disclosed in PCT publication number WO 94/00153 (the entire contents of which are incorporated herein by reference), or a less reactogenic composition where the QS21 is quenched with cholesterol as disclosed in PCT publication number WO 96/33739 (the entire contents of which are incorporated herein by reference). A particularly potent adjuvant formulation involving QS21 3D-MPL & tocopherol in an oil in water emulsion is described in PCT publication number WO 95/17210 (the entire contents of which are incorporated herein by reference). In addition, the nucleic acid of this invention can include an adjuvant by comprising a nucleotide sequence encoding a reagent of this invention and a nucleotide sequence that provides an adjuvant function, such as CpG sequences. Such CpG sequences, or motifs, are well known in the art.

An adjuvant of this invention, such as, for example, an immunostimulatory cytokine, can be administered before, concurrent with, and/or within a few hours, several hours, and/or 1, 2, 3, 4, 5, 6, 7, 8, 9, and/or 10 days or even weeks before or after the administration of a reagent of this invention to a subject.

Furthermore, any combination of adjuvants, such as immunostimulatory cytokines, can be co-administered to the subject before, after or concurrent with the administration of a reagent of this invention. For example, combinations of immunostimulatory cytokines, can consist of two or more immunostimulatory cytokines of this invention, such as GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules. The effectiveness of an adjuvant or combination of adjuvants can be determined by measuring the immune response directed produced in response to administration of a reagent of this invention to a subject with and without the adjuvant or combination of adjuvants, using standard procedures, as described herein and as known in the art.

Pharmaceutical compositions comprising a reagent of this invention and a pharmaceutically acceptable carrier are also provided. The compositions described herein can be formulated for administration in a pharmaceutical carrier in accordance with known techniques. See, e.g., Remington, The Science And Practice of Pharmacy (latest edition). In the manufacture of a pharmaceutical composition according to embodiments of the present invention, the composition of this invention is typically admixed with, inter alia, a pharmaceutically acceptable carrier. By “pharmaceutically acceptable carrier” is meant a carrier that is compatible with other ingredients in the pharmaceutical composition and that is not harmful or deleterious to the subject. The carrier may be a solid or a liquid, or both, and is preferably formulated with the composition of this invention as a unit-dose formulation. The pharmaceutical compositions are prepared by any of the well-known techniques of pharmacy including, but not limited to, admixing the components, optionally including one or more accessory ingredients.

The pharmaceutical compositions of this invention include those suitable for oral, rectal, topical, inhalation (e.g., via an aerosol) buccal (e.g., sub-lingual), vaginal, parenteral (e.g., subcutaneous, intramuscular, intradermal, intraarticular, intrapleural, intraperitoneal, intracerebral, intraarterial, or intravenous), topical (i.e., both skin and mucosal surfaces, including airway surfaces) and transdermal administration, although the most suitable route in any given case will depend, as is well known in the art, on such factors as the species, age, gender and overall condition of the subject, the nature and severity of the condition being treated and/or on the nature of the particular composition (i.e., dosage, formulation) that is being administered.

Pharmaceutical compositions suitable for oral administration can be presented in discrete units, such as capsules, cachets, lozenges, or tables, each containing a predetermined amount of the composition of this invention; as a powder or granules; as a solution or a suspension in an aqueous or non-aqueous liquid; or as an oil-in-water or water-in-oil emulsion. Oral delivery can be performed by complexing a composition of the present invention to a carrier capable of withstanding degradation by digestive enzymes in the gut of an animal. Examples of such carriers include plastic capsules or tablets, as known in the art. Such formulations are prepared by any suitable method of pharmacy, which includes the step of bringing into association the composition and a suitable carrier (which may contain one or more accessory ingredients as noted above). In general, the pharmaceutical composition according to embodiments of the present invention are prepared by uniformly and intimately admixing the composition with a liquid or finely divided solid carrier, or both, and then, if necessary, shaping the resulting mixture. For example, a tablet can be prepared by compressing or molding a powder or granules containing the composition, optionally with one or more accessory ingredients. Compressed tablets are prepared by compressing, in a suitable machine, the composition in a free-flowing form, such as a powder or granules optionally mixed with a binder, lubricant, inert diluent, and/or surface active/dispersing agent(s). Molded tablets are made by molding, in a suitable machine, the powdered compound moistened with an inert liquid binder.

Pharmaceutical compositions suitable for buccal (sub-lingual) administration include lozenges comprising the composition of this invention in a flavored base, usually sucrose and acacia or tragacanth; and pastilles comprising the composition in an inert base such as gelatin and glycerin or sucrose and acacia.

Pharmaceutical compositions of this invention suitable for parenteral administration can comprise sterile aqueous and non-aqueous injection solutions of the composition of this invention, which preparations are preferably isotonic with the blood of the intended recipient. These preparations can contain anti-oxidants, buffers, bacteriostats and solutes, which render the composition isotonic with the blood of the intended recipient. Aqueous and non-aqueous sterile suspensions, solutions and emulsions can include suspending agents and thickening agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The compositions can be presented in unit\dose or multi-dose containers, for example, in sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, an injectable, stable, sterile composition of this invention in a unit dosage form in a sealed container can be provided. The composition can be provided in the form of a lyophilizate, which can be reconstituted with a suitable pharmaceutically acceptable carrier to form a liquid composition suitable for injection into a subject. When the composition is substantially water-insoluble, a sufficient amount of emulsifying agent, which is physiologically acceptable, can be included in sufficient quantity to emulsify the composition in an aqueous carrier. One such useful emulsifying agent is phosphatidyl choline.

Pharmaceutical compositions suitable for rectal administration are preferably presented as unit dose suppositories. These can be prepared by admixing the composition with one or more conventional solid carriers, such as for example, cocoa butter and then shaping the resulting mixture.

Pharmaceutical compositions of this invention suitable for topical application to the skin preferably take the form of an ointment, cream, lotion, paste, gel, spray, aerosol, or oil. Carriers that can be used include, but are not limited to, petroleum jelly, lanoline, polyethylene glycols, alcohols, transdermal enhancers, and combinations of two or more thereof. In some embodiments, for example, topical delivery can be performed by mixing a pharmaceutical composition of the present invention with a lipophilic reagent (e.g., DMSO) that is capable of passing into the skin.

Pharmaceutical compositions suitable for transdermal administration can be in the form of discrete patches adapted to remain in intimate contact with the epidermis of the subject for a prolonged period of time. Compositions suitable for transdermal administration can also be delivered by iontophoresis (see, for example, Pharmaceutical Research 3:318 (1986)) and typically take the form of an optionally buffered aqueous solution of the composition of this invention. Suitable formulations can comprise citrate or bis\tris buffer (pH 6) or ethanol/water and can contain from 0.1 to 0.2M active ingredient.

An effective amount of a composition of this invention, the use of which is in the scope of present invention, will vary from composition to composition, and subject to subject, and will depend upon a variety of well known factors such as the age, race, gender and condition of the subject and the form of the composition and route of delivery. An effective amount can be determined in accordance with routine pharmacological procedures known to those skilled in the art (see, e.g., Remington's Pharmaceutical Sciences, latest edition).

The compositions of this invention can be administered to a cell of a subject either in vivo or ex vivo. For administration to a cell of the subject in vivo, as well as for administration to the subject, the compositions of this invention can be administered, for example as noted above, orally, parenterally (e.g., intravenously), by intramuscular injection, intradermally (e.g., by gene gun), by intraperitoneal injection, subcutaneous injection, transdermally, extracorporeally, topically or the like. Also, the composition of this invention may be pulsed onto dendritic cells, which are isolated or grown from patient cells, according to methods well known in the art, or onto bulk PBMC or various cell subfractions thereof from a patient.

If ex vivo methods are employed, cells or tissues can be removed and maintained outside the body according to standard protocols well known in the art while the compositions of this invention are introduced into the cells or tissues. For example, the nucleic acids and vectors of this invention can be introduced into cells via any gene transfer mechanism, such as, for example, virus-mediated gene delivery, calcium phosphate mediated gene delivery, electroporation, microinjection or proteoliposomes. The transduced cells can then be infused (e.g., in a pharmaceutically acceptable carrier) or transplanted back into the subject per standard methods for the cell or tissue type. Standard methods are known for transplantation or infusion of various cells into a subject. Thus, in one embodiment of this invention, the chimeric polypeptide of this invention can be presented to the immune system in a subject on the surface of a cell (i.e., as a cell surface antigen present in the plasma membrane of the cell) and in other embodiments can be presented to the immune system in a subject as a non-cell associated (i.e., cell-free) chimeric polypeptide.

Administration of the nucleic acids of this invention can be achieved by any one of numerous, well-known approaches, for example, but not limited to, direct transfer of the nucleic acids, in a plasmid or viral vector, or via transfer in cells or in combination with carriers such as cationic liposomes. Such methods are well known in the art and readily adaptable for use in the methods described herein. Furthermore, these methods can be used to target certain diseases and cell populations by using the targeting characteristics of the carrier, which would be well known to the skilled artisan.

Transfer vectors employed in the methods of this invention can be any nucleotide construct used to deliver nucleic acid into cells, e.g., a plasmid or viral vector, such as a retroviral vector which can package a recombinant retroviral genome (see e.g., Pastan et al., Proc. Natl. Acad. Sci. U.S.A. 85:4486 (1988); Miller et al., Mol. Cell. Biol. 6:2895 (1986)). The recombinant retrovirus can then be used to infect and thereby deliver a nucleic acid of the invention to the infected cells. The exact method of introducing the nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naldini et al., Science 272:263-267, 1996), pseudotyped retroviral vectors (Agrawal et al., Exper. Hematol. 24:738-747, 1996), and any other vector system now known or later identified. Physical transduction techniques can also be used, such as liposome delivery and receptor-mediated and other endocytosis mechanisms (see, for example, Schwartzenberger et al., Blood 87:472-478, 1996). This invention can be used in conjunction with any of these or other commonly used nucleic acid transfer methods. Appropriate means for transfection, including viral vectors, chemical transfectants, or physico-mechanical methods such as electroporation and direct diffusion of DNA, are described by, for example, Wolff et al., Science 247:1465-1468, (1990); and Wolff., Nature 352:815-818, (1991).

As noted above, the compositions of this invention can be used in various methods to enhance an immune response and/or to treat or prevent a cancer and/or disease or disorder in a subject.

“Effective amount” refers to an amount of a reagent or composition of this invention that is sufficient to produce a desired effect, which can be a therapeutic effect. The effective amount will vary with the age, gender, race, species, general condition, etc., of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an “effective amount” in any individual case can be determined by one of ordinary skill in the art by reference to the pertinent texts and literature and/or by using routine experimentation. (See, for example, Remington, The Science And Practice of Pharmacy (20th ed. 2000)).

“Treat,” “treating” or “treatment” refers to any type of action that imparts a modulating effect, which, for example, can be a beneficial effect, to a subject afflicted with a disorder, disease or illness, including improvement in the condition of the subject (e.g., in one or more symptoms), delay in the progression of the condition, prevention or delay of the onset of the disorder, and/or change in clinical parameters, disease or illness, etc., as would be well known in the art.

A “subject in need thereof” is a subject known to be, or suspected of having cancer or of having an infection as described herein. A subject of this invention can also include a subject not previously known or suspected to have cancer or an infection or in need of treatment for cancer or infection. For example, a subject of this invention can be administered the compositions of this invention even if it is not known or suspected that the subject has cancer or an infection (e.g., prophylactically). A subject of this invention is also a subject known or believed to be at risk of cancer or infection.

The disease and/or disorder that can be treated by the methods of this invention can include any disease or disorder that can be treated by mounting an effective immune response to an antigen of this invention, as well as any disease or disorder that can be treated by enhancing an immune response to an antigen of this invention by suppressing regulatory immune cells in a subject. For example, the methods of the present invention can be used to treat cancer, viral infections, bacterial infections, fungal infections, parasitic infections and/or other diseases and disorders that can be treated by eliciting an immune response in a subject of this invention.

It is also contemplated that the compositions of this invention can be used as a vaccine or prophylactic composition and employed in methods of preventing a disease or disorder in a subject, comprising administering to the subject an effective amount of the composition of this invention. The vaccine can be administered to a subject who is identified to be at risk of contracting a particular disease or developing a particular disorder and in whom the ability to elicit an immune response to an antigen may be impaired. Identification of a subject at risk can include, for example, evaluation of such factors as family history, genetic predisposition, age, environmental exposure, occupation, lifestyle and the like, as are well known in the art.

The present invention additionally provides kits comprising a first reagent for reducing or eliminating an immunosuppressive activity of a cell in a subject and a second reagent for treating and/or preventing cancer and/or an infectious disease or disorder in a subject, with or without an adjuvant, along with appropriate buffers, diluents, vessels and/or devices, etc. for measuring a specific amount and for administering the compositions to a subject of this invention. An example of a kit of this invention includes a fusion protein comprising a targeting moiety and a toxic moiety (e.g., ONTAK™) as a first reagent and dendritic cells loaded with an antigen specific for a tumor of a specific subject as a second reagent. Another example includes ONTAK™ as a first reagent and an HCV antigen as a second reagent. Numerous other examples are encompassed within the scope of this invention, as would be well recognized by one of skill in the art.

### EXAMPLES

### Example 1

As one embodiment of the present invention, the recombinant fusion protein denileukin diftitox (DAB₃₈₉IL-2) was used to eliminate or functionally inactivate CD25-expressing regulatory T cells in vitro and in vivo. DAB₃₈₉IL-2 contains the catalytic and membrane translocation domain of diphtheria toxin. The binding domain for the diphtheria toxin receptor, however, is deleted and replaced by the human IL-2 gene, which allows for targeting of CD25-expressing cells. The cytotoxic action of DAB₃₈₉IL-2 occurs as a result of binding to the IL-2 receptor (IL-2R), subsequent internalization and enzymatic inhibition of protein synthesis leading to cell death.

The present studies show that DAB₃₈₉IL-2 is capable of selectively eliminating or reducing regulatory T cell subsets from PBMC in a dose-dependent manner without bystander toxicity to other PBMC or to CD4⁺ T cells with intermediate or low-level expression of CD25. Regulatory T cell depletion resulted in enhanced stimulation of proliferative and cytotoxic T-cell responses in vitro, but only when DAB₃₈₉IL-2 was used prior to and omitted during the T cell priming phase.

Depletion of regulatory T cells in cancer patients followed by vaccination with tumor RNA-transfected DC resulted in improved stimulation of tumor-specific CTL when compared to vaccination alone. This study provides the first clinical evidence that in vivo elimination of regulatory T cell subsets can enhance the magnitude of vaccine-mediated, tumor-specific T-cell responses.

Clinical Trial Design and Patient Eligibility

This study was initiated as a randomized 2×2 multifactorial design, enrolling patients with metastatic renal cell carcinoma. This report provides results of the first eight patients enrolled on this protocol. Treatment of patients was performed following written informed consent on an Institutional Review Board-approved protocol. Patients with histologically-confirmed metastatic renal cell carcinoma were eligible for this study. One patient with stage IV metastatic ovarian carcinoma was included and treated on a compassionate basis protocol. All patients were required to have adequate hepatic, renal, and neurological function, a life expectancy of >6 months, and a Karnofsky performance status>70%. Patients must have had recovered from all toxicities related to any prior therapy and not have received any chemotherapy, radiation therapy, or immunotherapy for at least 6 weeks prior to study entry. Excluded from the study were patients with central nervous system metastases, patients with a history of autoimmune disease, with serious intercurrent chronic or acute illnesses, or with concurrent second malignancy other than non-melanoma skin cancer, or controlled superficial bladder cancer. Patients on steroid therapy or other immunosuppressive agents were also excluded.

According to the clinical trial protocol, eligible subjects were randomly assigned to receive either a single dose of DAB₃₈₉IL-2 (18 μg/kg) followed by vaccination with tumor RNA-transfected DC (treatment arm A) or to vaccination alone (treatment arm B). All subjects received a total of three intradermal injections of total tumor RNA-transfected DC. The injections were given intradermally at biweekly intervals and consisted of 1×10⁷ cells suspended in 200 μL 0.9% sodium chloride (Abbott Laboratories, Abbott Park, Ill.) at each vaccination cycle. Following treatment, subjects were evaluated for toxicity, immunological and clinical response to therapy. Follow-up visits occurred biweekly for three visits, monthly for one visit, then every 3 months or until the subject was removed from the study.

DAB₃₈₉IL-2 and Vaccine Preparation

DAB₃₈₉IL-2 was provided by Ligand Pharmaceuticals San Diego, Calif. as a frozen, sterile solution formulated in citrate buffer in 2 ml single use vials at a concentration of 150 μg/ml. After thawing, DAB₃₈₉IL-2 was diluted with sterile normal saline to a final concentration of 15 μg/ml and delivered by intravenous infusion over a 30-minute period. Patients were permitted to receive acetaminophen (600 mg) and antihistamines 30 to 60 minutes prior to infusion.

Dendritic cells were manufactured in a dedicated cell processing facility using standardized, Food and Drug Administration-approved protocols. For DC culture, a concentrated leukocyte fraction was harvested by leukapheresis. Peripheral blood mononuclear cells (PBMC) were isolated from the leukapheresis product by density gradient centrifugation over polysucrose/sodium diatrizoate (HISTOPAQUE®, Sigma Diagnostics, St. Louis, Mo.) and cells were resuspended in serum-free AIMV™ medium (GIBCO BRL, Grand Island, N.Y.). PBMC were incubated in a humidified incubator for two hours at 37° C. to allow plastic adherence. The semi-adherent cell fraction was used for DC culture by incubation in serum-free X-VIVO 15™ medium (Cambrex Bio Science, Walkersville, Md.) supplemented with rhIL-4 (500 U/ml) (R&D Systems, Minneapolis, Minn.) and rhGM-CSF (800 U/ml) (Immunex, Seattle, Wash.) After 7 days of culture, cells were harvested and used for mRNA transfection.

Total RNA was isolated using the RNeasy kit (Qiagen, Valencia, Calif.) from primary tumor tissues histologically classified as clear cell carcinoma. All RNA preparations were subjected to electrophoresis in 1.2% agarose gel under denaturing conditions with clear visualization of 18S and 28S ribosomal bands following ethidium bromide staining. Control RNA used for immunological monitoring studies was extracted from autologous benign renal tissues (RE) or from PBMC.

Immature DC were transfected with total tumor RNA by electroporation. In brief, DC were washed twice in phosphate-buffered saline, counted, and resuspended at a concentration of 4×10⁷ cells/ml in ViaSpan® (Barr Laboratories Inc., Pomona, N.Y.). Cells were then coincubated for 5 minutes with 5 μg RNA per 1×10⁶ cells on ice and electroporated in 0.4 cm cuvettes via exponential decay delivery at 300V and 150 μF (Gene Pulser II, BioRad, Hercules, Calif.). After electroporation, cells were centrifuged, resuspended in X-VIVO 15™ medium, and matured for 20 hours in the presence of 10 ng/ml TNF-α, 10 ng/ml IL-1β, 150 ng/ml IL-6 (R&D Systems, Minneapolis, Minn.), and 1 μg/ml PGE₂ (Cayman Chemicals, Ann Arbor, Mich.). Prior to administration, cells were phenotypically characterized to ensure that they met the typical phenotype of fully mature DCs: Lin^{neg}, HLA class I and II^{high}, CD86^{high}, CD83^{high}.

Evaluation of Immune Status

Interferon-γ and IL-4 ELISPOT analyses were performed using PBMC obtained prior to, during, and after vaccination. PBMC were cultured overnight in RPMI 1640 medium supplemented with 10% FCS. CD4⁺ and CD8⁺ T cells were isolated from PBMC by magnetic bead-based negative depletion (Miltenyi, Bergisch-Gladbach, Germany). After blocking wells with complete medium, 1×10⁵ T cells and 1×10⁴ RNA-transfected DC in 100 μl of complete medium were added to each well of flat-bottomed 96-well nitrocellulose plates (Multiscreen-IP, Millipore, Bedford, Mass.) precoated with 2 μg/ml Interferon-γ capture antibody (Endogen, Rockford, Ill.) or with IL-4 capture antibody according to the manufacturer's recommendations (BD Biosciences Pharmingen, San Diego, Calif.). Plates were incubated for 20 hours at 37° C., and biotinylated Interferon-γ detection antibody (Endogen, Rockford, Ill.) or biotinylated IL-4 antibody (BD Biosciences Pharmingen, San Diego, Calif.) was added to each well. Cells were then incubated for an additional 2 hours at room temperature, then with streptavidin-alkaline phosphatase (1 μg/ml; Sigma, St. Louis, Mo.) and plates were developed with substrate (Kirkegaard & Perry Laboratories, Gaithersburg, Md.). After washing, spots were counted using an automated Zeiss KS Elispot Compact reader (Carl Zeiss Inc., Minneapolis, Minn.).

CTL assays were performed by co-culturing the RNA transfected DC with autologous PBMC at a DC:PBMC ratio of 1:10. Cells were restimulated once and IL-2 (20 units/ml) was added after 5 days and every other day thereafter. After 12 days of culture, effector cells were harvested for cytolytic assays. Target cells were labeled with 100 μCi of Na₂[⁵¹CrO₄] (NEN, Boston, Mass.) in 200 μl of complete RPMI 1640 for 1 hour at 37° C. in 5% CO₂ and ⁵¹Cr-labeled target cells were incubated in complete RPMI 1640 medium with effector cells for 5 hours at 37° C. Then, 50 μl of supernatant was harvested, and release of ⁵¹Cr was measured with a scintillation counter. Results from triplicate wells were averaged, and the percentage of specific lysis was calculated.

For proliferation assays, purified CD3⁺ T cells were seeded into 96-well round-bottomed microplates in the presence of mRNA-transfected DC. Triplicate wells of T cells alone were used as the background control. After 4 days of culture, 1 μCi of [methyl-3H]thymidine (NEN, Boston, Mass.) was added to each well, and incubation was continued for an additional 16 hours. Cells were collected onto glass fiber filters (Wallac, Turku, Finland) with a cell harvester, and incorporation of thymidine into DNA was determined using a liquid scintillation counter.

Cytotoxocity of DAB₃₈₉IL-2 was determined in MTT (3-(4,5-dimethylthazol-2-yl)-2,5-diphenyl tetrazolium bromide salt) assays. After a 6-hour incubation with varying concentrations of DAB₃₈₉IL-2, cells were seeded in triplicate in 96-well plates in 100 μL complete media at a density of 5×10³ cells/well. After 48 hours of incubation, 20 μL MTT from a 5 mg/mL stock was added and incubation was continued for another 4 hours. The formazan crystals were solubilized by adding 100 μL isopropanol/0.1 M hydrochloric acid and incubating at 37° C. for 2 hours. The absorbance of the formazan product was measured on an enzyme-linked immunosorbent assay (ELISA) plate reader at 570 nm.

Fluorescence-Activated Cell Sorter (FACS) Analysis

Four-color FACS analyses were performed using a Becton Dickinson FACSCalibur™. Fluorochrome-conjugated antibodies including anti-CD4 FITC, anti-CD45RO, anti-CD45RA (Caltag, Burlingame, Calif.); anti-CD25 PE (Becton Dickinson, California, CA) as well as isotypic control antibodies (Caltag, Burlingame, Calif.) were used for T-cell staining. Expression of GITR was analyzed by staining T cells with anti-GITR antibody (R&D Systems, Minneapolis, Minn.) followed by secondary goat anti-mouse antibody conjugated to APC. For determination of intracellular CTLA-4 expression, T cells were permeabilized with 0.5% saponin, fixed with 4% paraformaldehyde and then stained with biotinylated anti-CD152 (Becton Dickinson, California, CA) followed by APC-strepavidin (Becton Dickinson, California, CA). A total of 1×10⁶ cells were suspended in staining buffer (PBS with 1% FCS, 2 mM EDTA, and 0.1% sodium azide) and incubated for 20 minutes at 4° C. with the antibody. Data were analyzed and presented using CELLQuest™ software. Sorting of CD4⁺CD25^{neg}, CD4⁺CD25^{int} and CD4⁺CD25^{high} T cells was performed with a BD FACSAria™ cell sorter after antibody labeling. For detection of cytokine secretion, isolated CD4⁺ T cells were activated for 16 hours in the presence of autologous mRNA-transfected DC. Cytokine secretion was measured using c Th1/Th2 cytokine cytometric bead arrays according to the manufacturer's protocol (BD Biosciences Pharmingen, San Diego, Calif.).

Phenotypic and Functional Characterization of Regulatory T Cells

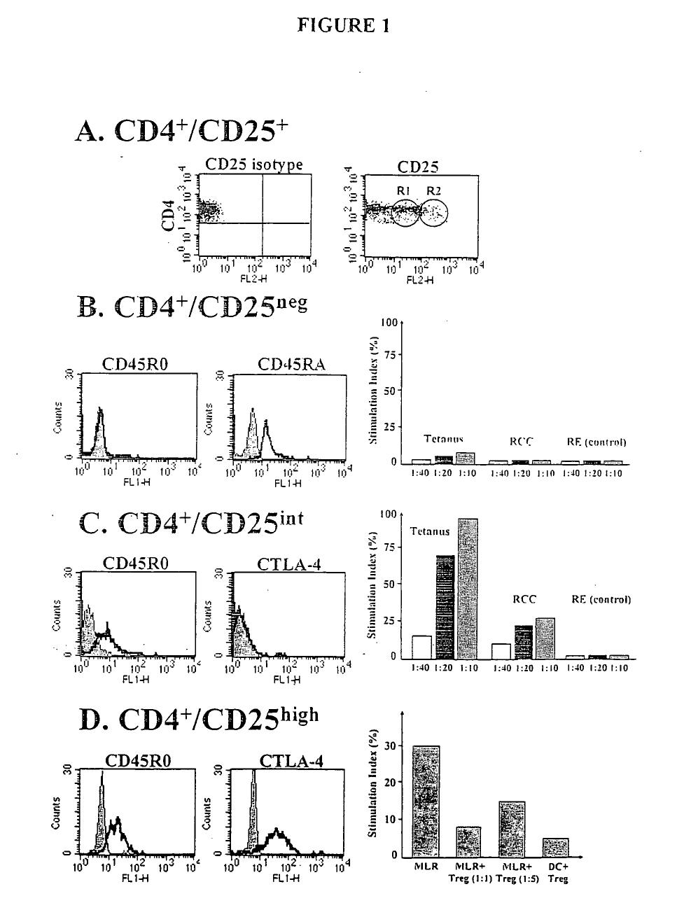

Unlike in mice, the definition and enumeration of regulatory T cells in humans is complicated by the fact that CD25 is a marker of T cell activation and effector T-cell function. In fact, human CD4⁺ T cells expressing CD25 represent a heterogeneous cell population containing not only regulatory, but also effector/memory T cells. Analysis of PBMC from healthy volunteers and cancer patients revealed the presence of CD4⁺ T cell populations expressing increasing levels of CD25, as shown previously (FIG. 1A). While one population of CD4⁺ cells lacked CD25 expression, another subset exhibited intermediate levels (R1), and a third, albeit small portion expressed high cell surface expression of CD25 (R2). To phenotypically and functionally characterize these three distinct subsets, CD4⁺CD25⁻, CD4⁺CD25^{int}, and CD4⁺CD25^{high} T cells were isolated from PBMC by FACS. CD4⁺CD25⁻ cells expressed cell surface markers characteristic of naïve/resting T cells (CD45RO⁻, CD45RA⁺). Accordingly, stimulation of naïve CD4⁺/CD25⁻ T cells with DC loaded with a) tetanus toxoid (Tetanus) or b) transfected with RNA from renal tumors (RCC), c) benign renal epithelial (RE), or d) PBMC, resulted in only minor reactivities in proliferation assays (FIG. 1B). In contrast, CD4⁺CD25^{int} cells exhibiting a typical effector/memory T cell phenotype CD45RO^{-/+}, CTLA-4⁻, and CD69⁺, exhibited a strong proliferative response following exposure to tetanus toxoid, and a lower, but significant response against RCC RNA-transfected DC. In contrast, no proliferative response against RE or PBMC RNA-transfected DC (control) was observed (FIG. 1C).

CD4⁺/CD25^{high} T-cells were consistently positive for CD45RO, and constitutively expressed intracellular CTLA-4, consistent with a phenotype characteristic for regulatory T cells (FIG. 1D, left panel). Accordingly, these cells exhibited immunosuppressive activity, as evidenced by a significant inhibition of mature, allogeneic DC-stimulated mixed lymphocyte reaction (MLR) cultures. As shown in FIG. 1D, right panel, the addition of increasing numbers of CD4⁺/CD25^{high} cells (1/5 responder cells; 1/1 responder cells) to MLR reactions led to a dose-dependent inhibition of responder cell proliferation, while CD4⁺/CD25^{high} T-cell did not proliferate significantly upon stimulation with mature DC (DC+Treg).

These studies show that only CD4⁺ T cells with high CD25 cell surface expression have suppressive activity and constitutively express CTLA-4, whereas CD4⁺ cells with intermediate expression levels are mainly comprised of T-cell subsets that provide immunological memory against infectious diseases and tumors.

Selective Elimination of Regulatory T Cells In Vitro

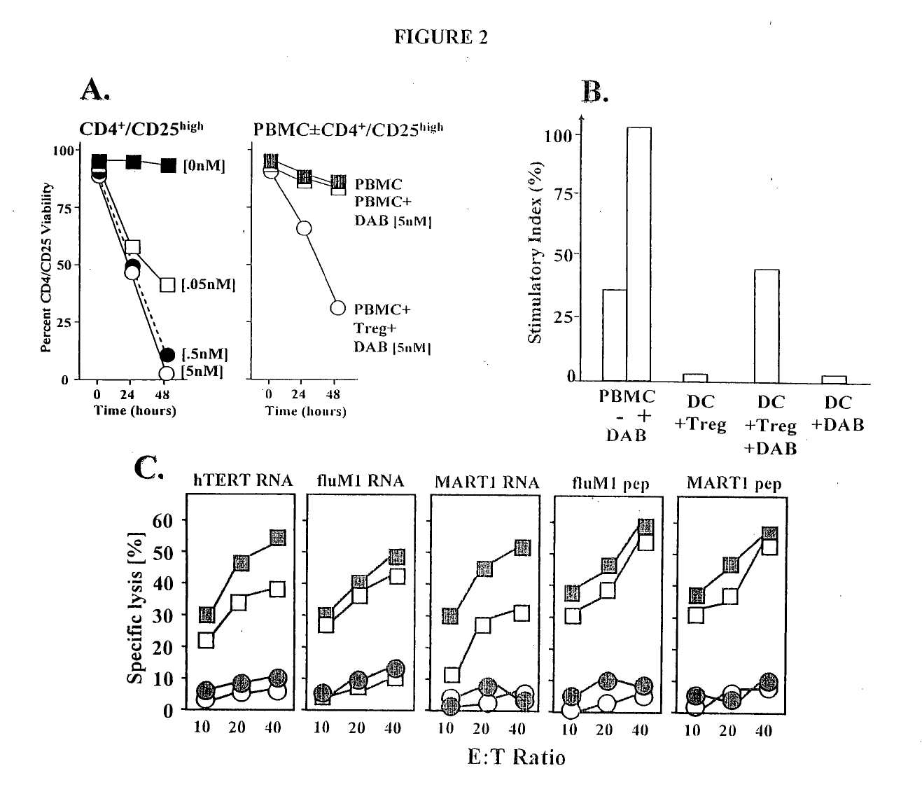

In humans, malignant hematopoietic cells expressing CD25 can be eliminated or functionally inactivated using the recombinant IL-2 diphtheria toxin conjugate, denileukin difititox (DAB₃₈₉IL-2). In order to determine whether DAB₃₈₉IL-2 could serve as a suitable reagent to achieve CD4⁺/CD25^{high} regulatory T-cell depletion under clinically relevant conditions, regulatory T cell susceptibility to DAB₃₈₉IL-2 was analyzed in MTT assays. In these experiments, conditions were chosen that resembled the pharmacokinetics of a single, intravenous dose of DAB₃₈₉IL-2 (18 μg/kg), which, by calculation, results in 5 nM peak plasma concentrations. Given a plasma half-life of 54 minutes and a dissociation constant of 1 pM for DAB₃₈₉IL-2 and the high-affinity IL-2 receptor, DAB₃₈₉IL-2 plasma levels were projected to reach suboptimal concentrations after 6 hours (DAB₃₈₉IL-2-IL-2 receptor interactions no longer follow pseudo-first order kinetics). Therefore, the viability of isolated CD4⁺CD25^{high} T cells was analyzed after a 6-hour exposure to increasing concentrations of DAB₃₈₉IL-2 (range 0.05-5 nM) in vitro over 48 hours (FIG. 2A). For CD4⁺CD25^{high} regulatory T cells, a significant reduction in cell viability was observed 24 hours following exposure with DAB₃₈₉IL-2. Efficient killing of CD4⁺CD25^{high} cells was observed at 0.5 nM concentrations, while complete depletion was achieved at 5 nM. In contrast, exposure of CD4⁺CD25⁻ and CD4⁺CD25^{int} cells to DAB₃₈₉IL-2 did not result in significant cell death, except when cells were exposed to concentrations of DAB₃₈₉IL-2 higher than 10 nM. In another set of experiments studies were conducted to determine whether DAB₃₈₉IL-2, used at a 5 nM concentration, resulted in specific killing of regulatory T cells, but not of other bystander cells in vitro. As shown in FIG. 2A, right panel, there were no significant differences in PBMC viability over time when DAB₃₈₉IL-2 [5 nM] was added to the culture. In contrast, when PBMC and regulatory T cells were mixed at a 1: I ratio, cell viability was reduced by more than 60% at 48 hours, indicating selective regulatory T cell killing without any bystander cell toxicity mediated by diphtheria toxin or its toxic metabolites. Next, the impact of DAB₃₈₉IL-2 on freshly activated lymphocytes after stimulation with mature, allogeneic DC in mixed lymphocyte reactions (MLR) was determined. As shown in FIG. 2B, DAB₃₈₉IL-2-mediated depletion of regulatory T cells prior to initiation of the MLR culture resulted in a 2-fold increase in proliferation of responder cells (PBMC±DAB), while, conversely, the addition of isolated regulatory T cells (DC+Treg) resulted in an approximately 80% reduction of T cell proliferation (2:1 T cell to responder ratio). Pre-incubation of regulatory T cells with DAB₃₈₉IL-2 [5 nM] could significantly abrogate their inhibitory effect when added to the MLR reaction, however, responder cells did not proliferate as vigorously as in the absence of regulatory T cells, indicating potential contact inhibition by regulatory T cells. Importantly, the addition of DAB₃₈₉IL-2 two days after initiation of the MLR reaction (DC+DAB) completely abrogated the proliferation of responder cells, demonstrating that DAB₃₈₉IL-2 does not only eliminate regulatory T cells, but also CD25-expressing, freshly activated naive T cells.

In summary, these experiments demonstrate that DAB₃₈₉IL-2 is a suitable reagent for selectively eliminating regulatory T cells in vitro without affecting other lymphocyte subsets, including naïve and memory T cells expressing low to intermediate levels of CD25. These data further show that DAB₃₈₉IL-2 can only be applied prior to immunization, but not during the vaccination (T cell priming) phase.

Enhancement of T-Cell Responses after Regulatory T-Cell Depletion

To provide evidence that DAB₃₈₉IL-2-mediated regulatory T cell depletion is capable of augmenting antigen-specific T cell responses in vitro, CTL from DAB₃₈₉IL-2-depleted and non-depleted PBMC were generated (FIG. 2C). PBMC were stimulated twice with autologous dendritic cells transfected with telomerase (hTERT), influenza matrix protein-1 (fluM1), and MART-1 mRNA (control). In addition, DC pulsed with HLA-A0201-restricted fluM1- or MART-1 peptides were used as stimulators. RNA-transfected DC were not only used as stimulators but also served as specific or control targets, as shown previously. The ability of the stimulated, antigen-specific CTLs to recognize their cognate, but not control targets cells were analyzed in standard cytotoxicity assays.

As shown in FIG. 2C, CTLs stimulated from regulatory T cell-depleted PBMCs consistently exhibited higher lytic activity against their cognate targets than CTLs stimulated from non-depleted peripheral blood. This improvement was reduced when fluM1 mRNA transfected DC, or peptide-pulsed DC were used as stimulators.

These data show that depletion of regulatory T cells from peripheral blood results in enhancing the stimulation of antigen-specific CTL in vitro. It appears that the impact of this strategy is most pronounced in potentiation T cell responses against naturally-processed self-antigens such as hTERT and MART-1. In contrast, the regulatory T cell depletion strategy was ineffective in improving CTL responses against the recall antigen fluM1, or when peptide-loaded DCs were used for stimulation.

Depletion of Regulatory T Cells In Vivo

Having shown that regulatory T cell elimination is capable of enhancing T-cell responses in vitro, a clinical study was initiated to analyze the efficacy of this strategy to a) eliminate regulatory T cells and b) to enhance a vaccine-induced T-cell response in end-stage cancer patients. Seven patients with metastatic renal cell (RCC), and one subject with disseminated ovarian carcinoma (OVA) were treated in an institutional review board (IRB) and Food and Drug Administration (FDA)-approved pilot study. Four subjects received a single intravenous dose of DAB₃₈₉IL-2 (18 μg/kg) 4 days prior to vaccination with tumor RNA transfected DC, while a second cohort of four subjects was treated with the vaccine alone. PBMCs were also obtained from a subject who received DAB₃₈₉IL-2 only under separate informed consent. A detailed description of patient characteristics and treatment assignments is provided in Table 1. Toxicities after DAB₃₈₉IL-2 administration included Grade I constitutional symptoms in two subjects (HM-O₂; JVG-03) and transient, grade II ALT elevations in one subject (HM-02), as previously described. RNA-transfected DC injections were well tolerated without any major clinical toxicity and serologic/immunologic evidence of autoimmunity.

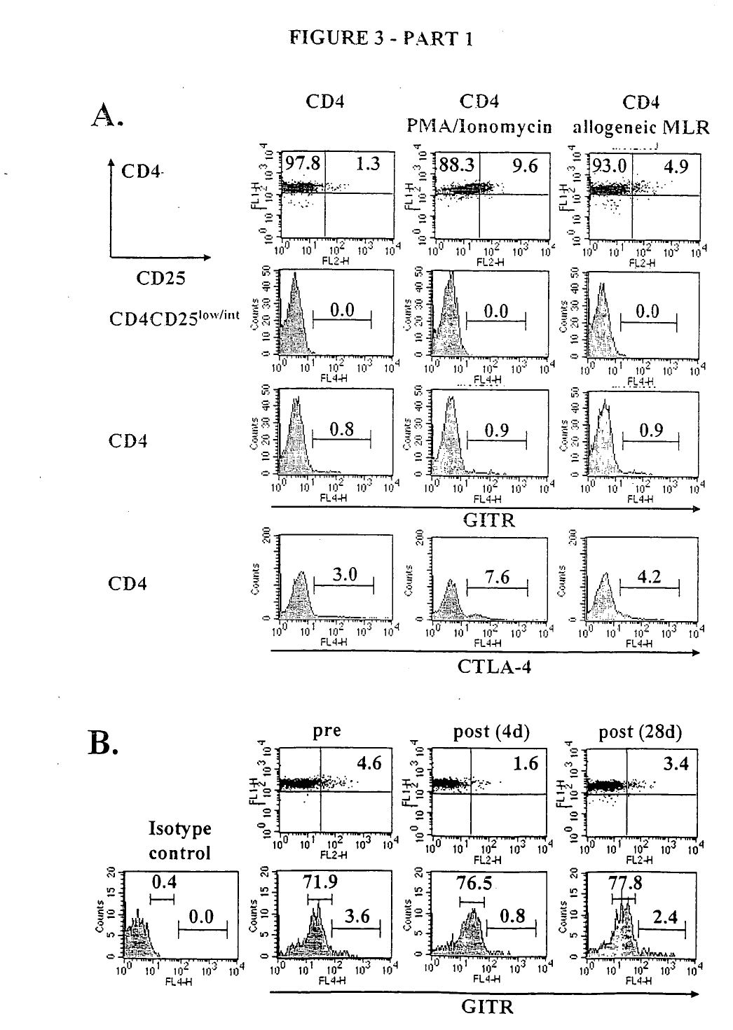

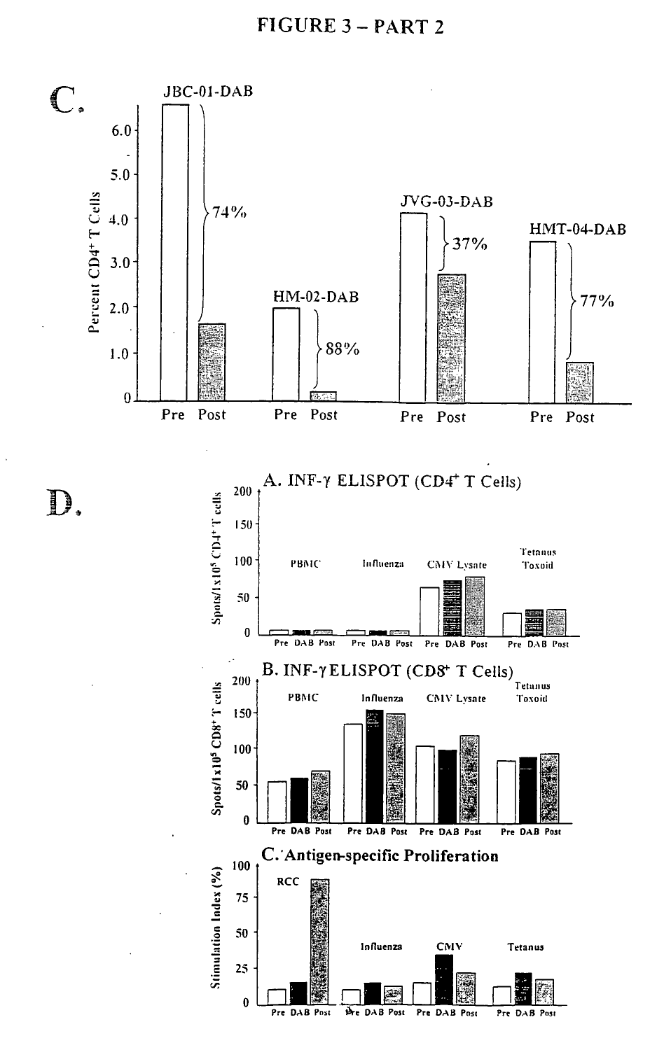

The feasibility to enumerate CD4⁺ regulatory T cells after vaccination solely based on CD25 expression levels was also determined. Naive and memory T cells may upregulate the expression of CD25 in response to antigenic stimulation, and may, therefore, acquire the phenotype of CD4⁺CD25^{high} regulatory T cells. Therefore, changes in CD25 expression were analyzed following polyclonal stimulation of CD4⁺ T cells with PMA/ionomycin or after stimulation of the (naive) CD4⁺ T cell subset in an allogeneic MLR. In parallel, studies were also conducted to determine the expression of the regulatory T-cell markers GITR and CTLA-4 in response to PMA/ionomycin or upon allo-antigen encounter. After stimulation, a significant shift was observed, from the CD4⁺CD25⁻ or CD4⁺CD25^{int} cell populations to a CD4⁺ CD25^{high} phenotype (FIG. 3A, top panel). Similarly, the frequency of CTLA-4 expressing cells increased upon stimulation of CD4⁺ T cells (FIG. 3A, bottom panel). In contrast, no change in the frequency of GITR^{high} expressing cells was noted upon T-cell stimulation.

These results show that GITR, but not CTLA-4, represents a suitable phenotypic marker to determine regulatory T cell frequencies during vaccination based on CD25 expression. This indicates that for accurate enumeration of regulatory T cells, analyses should include only GITR-expressing cells that then can be further analyzed for CD4 and CD25 expression.

Using these methods, the degree of regulatory T cell depletion efficacy in the four subjects treated on the clinical protocol was determined. As shown in FIGS. 3B and C, depletion efficacy in the four treated patients was 74%, 88%, 37%, and 77%, respectively. In all subjects, there was no significant change in the relative number of CD3+, CD4+, CD8⁺ T cells, B cells (CD19), monocytes/macrophages (CD14), and NK cells prior to and four days after treatment. Furthermore, in one subject analyzed (JBC-01-ONT), no decrease in CD8⁺/CD25⁺ or in CD19⁺/CD25⁺ cells after DAB₃₈₉IL-2 administration was found. Clinically, effective depletion (>75%) was associated with the emergence of constitutional symptoms or changes in blood chemistry, whereas the patient with a rather modest depletion level did not exhibit any symptoms or changes in blood count or chemistry.

In order to address the concern that DAB₃₈₉IL-2-mediated regulatory T cell depletion could induce toxicity within the T cell/memory pool (CD4⁺CD25^{int}), a series of experiments analyzing CD4⁺CD25^{int} T cell function four days after DAB₃₈₉IL-2 administration were performed. As shown in FIG. 3D, antigen-specific proliferation assays revealed unchanged reactivities against antigens presented by a) RCC RNA transfected DC (RCC), b) fluM1 RNA c) CMV lysate or d) tetanus toxoid, indicating that the risk of depleting naïve or memory/effector T cells after DAB₃₈₉IL-2 treatment is rather low. Moreover, Interferon-γ ELISPOT analyses performed on CD4⁺ and CD8⁺ T cells that were isolated from PBMC of the same patient prior to (pre), after DAB₃₈₉IL-2 administration, and after three vaccination cycles (post), did not reveal any changes in the frequencies of memory cells for the prototype recall antigens flu and tetanus toxoid, or frequencies of effector/memory cells specific for recently encountered cognate antigens such as CMV lysate or RCC RNA throughout the treatment cycle.

These data show that administration of a single dose of DAB₃₈₉IL-2 results in a significant reduction of regulatory T cells in most cancer patients. The functional data further indicate that under these conditions, no negative impact on memory/effector T cells should be expected.

In Vivo Stimulation of Tumor-Specific T-Cell Responses

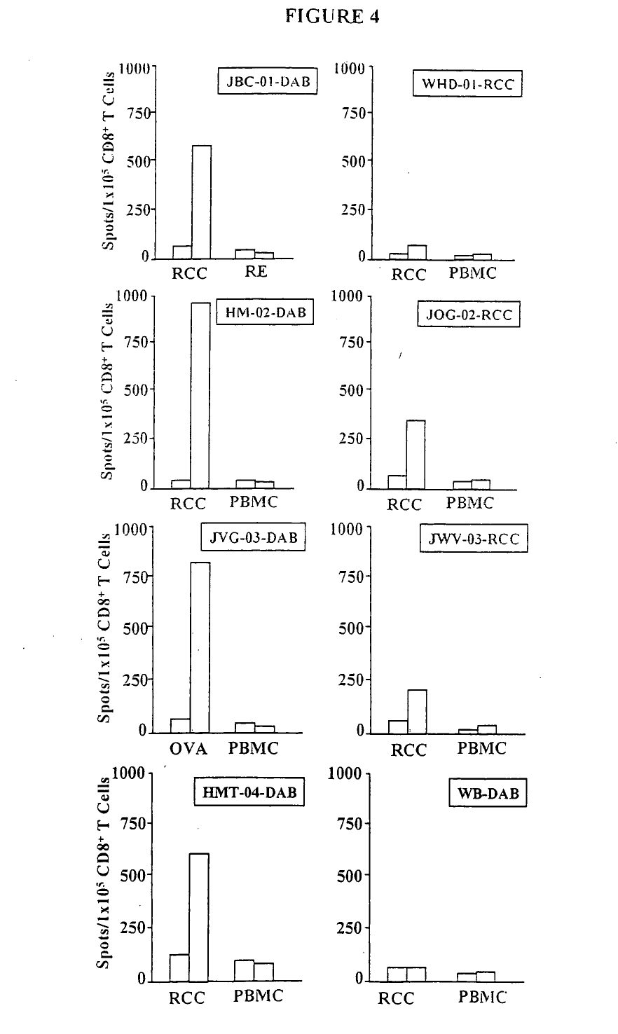

In order to determine whether DAB₃₈₉IL-2-mediated regulatory T cell depletion is capable of augmenting vaccine-induced CTL responses in vivo, Interferon-γ ELISPOT analysis was used to determine the frequencies of vaccine-induced, tumor-specific T cells from PBMC samples collected prior to (white bars) and two weeks after (grey bars) the third vaccination (study week 8). Purified CD8⁺ T cells were isolated from pre- and post-vaccination PBMCs and were cultured overnight with tumor RNA transfected DC targets. As controls, PBMC RNA or benign renal epithelium (RE)-derived RNA-transfected DCs were used for short-term antigenic stimulation. Visible spots were then counted using an automated ELISPOT reader. As shown in FIG. 4, only background levels of PBMC- or RE-specific T cells were observed in all subjects both prior to and after vaccination. Furthermore, treatment with DAB₃₈₉IL-2 alone did not increase a tumor-specific CTL response in vivo (JM-DAB). In contrast, a significant increase of tumor-specific T cells was consistently observed in the treated subjects after vaccination.

Although there was significant patient to patient variability in the magnitude of T cell responses measured in each patient, vaccination after regulatory T cell depletion stimulated significantly higher numbers of tumor-specific T cells (left panels) than treatment with the vaccine alone (right panels). The T− cell frequencies achieved after combined regulatory T cell depletion and three vaccination cycles were remarkably high with 0.6-1% of CD8⁺ T cells exhibiting tumor specificity.

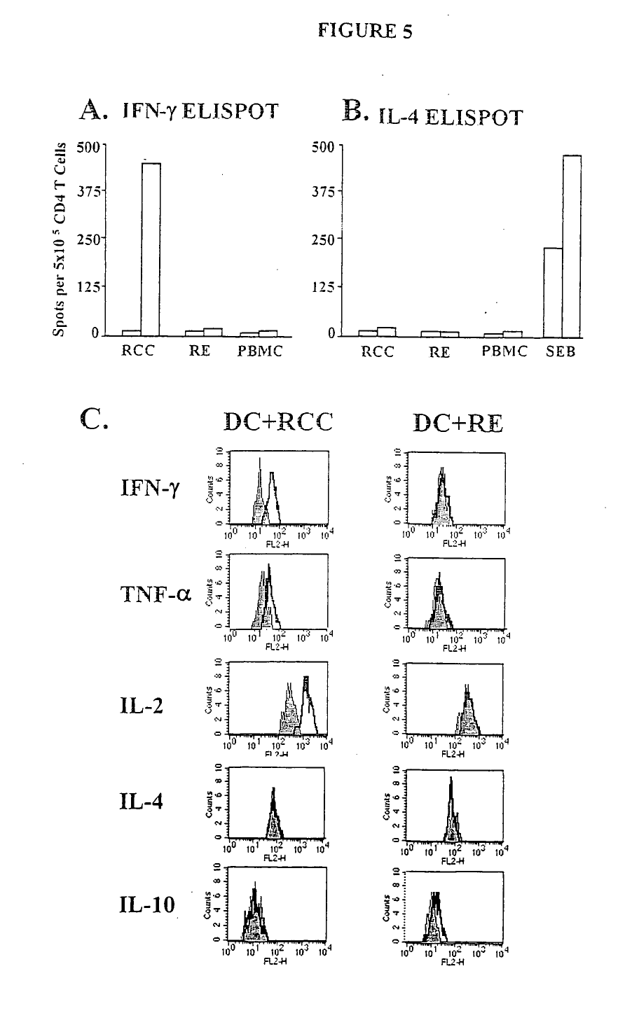

In one subject (JBC-01-DAB) from whom sufficient cell were available, studies were conducted to analyze whether not only tumor-specific CTL, but also CD4⁺ T cells were stimulated by treatment with DAB₃₈₉IL-2 followed by vaccination with renal tumor RNA-transfected DC. PBMC were collected at baseline (white bars) and two weeks after the final vaccination (grey bars) and CD4⁺ T cells were isolated by magnetic bead sorting. CD4⁺ T cells were re-stimulated for 18 hours with renal tumor RNA transfected DC and analyzed for Interferon-γ and IL-4 secretion using ELISPOT analysis. As control targets, influenza (flu) M1 mRNA-, renal epithelium (RE) RNA-transfected DC or SEB-loaded DC were used in these assays. As shown in FIG. 5A, vaccination after regulatory T cell depletion resulted in the stimulation of Interferon-γ, but not IL-4-secreting renal tumor-specific CD4 T cells. In addition, human Th1/Th2 flow-cytometric bead array assays revealed secretion of Th-1 type cytokines (IL-2, Interferon-γ, or TNF-α), but not Th-2 type cytokines (IL-10, IL-5 and IL-4) by vaccine-induced T cells after 18 hours of stimulation with RCC, but not RE RNA-transfected DC.

These studies provide clinical evidence that regulatory T cell depletion using the diphtheria fusion protein DAB₃₈₉IL-2 is capable of enhancing a vaccine-induced T cell response. Although in this pilot trial, a 2 to 10-fold increase of tumor-specific T cells could be measured after regulatory cell depletion and vaccination, whereas vaccine induced T-cell frequencies were similar to those observed in a prior study in which semi-mature tumor RNA transfected DC were used for vaccination.

The objective of this study was to enhance the immunostimulatory efficacy of RNA-transfected DC vaccines after selectively eliminating or reducing the numbers of CD4⁺CD25⁺⁺ regulatory T cells in cancer patients. Since this concept has not thus far been tested in a human vaccination setting, a series of preclinical studies were initiated to address the fundamental aspects of this strategy, followed by a clinical trial.

Described herein is a clinically applicable sequential protocol that entails regulatory T cell depletion using the anti-CD25 immunotoxin DAB₃₈₉IL-2, followed by vaccination using tumor RNA transfected DC. The impact of these strategies on the stimulation of a tumor-specific T cell response in cancer patients was studied. It was shown that human CD4⁺CD25^{high} regulatory T cells can be eliminated or reduced in a dose-dependent manner using clinically relevant doses without inducing bystander toxicity or impacting on the function of other cells expressing CD25. However, as shown in FIG. 2, DAB₃₈₉IL-2 exposure abrogated DC-mediated activation of T cells in vitro, indicating that the applicability of this reagent is restricted to a pre-vaccination setting.

These preclinical results provided important information on the design of the clinical study, in which DAB₃₈₉IL-2 was administered to cancer patients four days prior to vaccination with RNA transfected DC. This time interval was chosen since, unlike antibodies, DAB₃₈₉IL-2 is characterized by its short duration of action, with a half-life of minutes 60-90 minutes, thereby minimizing the possibility of interfering with the ongoing, vaccine-induced T cell response. Although there was considerable patient to patient variability in the magnitude of the observed T-cell response, higher numbers of tumor-specific T cells in the peripheral blood of DAB₃₈₉IL-2-treated subjects after vaccination were consistently observed, as compared to subjects who received the vaccine alone. The T-cell frequencies achieved after combined regulatory T cell depletion and three vaccination cycles were remarkable high, with 0.6-1% of all CD8⁺ T cells demonstrating tumor specificity.

The preclinical studies shown in FIG. 2 show that the regulatory T cell elimination strategy is predominantly geared towards the improvement of T-cell responses against (RNA-encoded) self-antigens such as hTERT or MART-1, but not against recall-, or peptide-derived antigens. Accordingly, it is shown that, if regulatory T cells were removed from PBMCs of patients vaccinated with renal tumor RNA transfected DC, reactivities against the self antigens OFA, G250 and hTERT, but not against fluM1, could be dramatically enhanced.

### Example 2

Clinical Trial Design and Patient Eligibility

Treatment of patients was performed following written informed consent as part of an Institutional Review Board- and Federal Drug Administration-approved protocol. Patients with histologically-confirmed metastatic RCC were eligible for this study. One patient with disseminated ovarian carcinoma was included and treated on a compassionate basis. All patients were required to have adequate hepatic, renal, and neurological function, a life expectancy of >6 months, and a Karnofsky performance status of >70%. Patients must have had recovered from all toxicities related to any prior therapy and have not received any chemotherapy, radiation therapy, or immunotherapy for at least 6 weeks prior to study entry. Excluded from the study were patients with CNS metastases, with a history of autoimmune disease, with serious intercurrent chronic or acute illnesses. Patients on immunosuppressive agents were also excluded. Eligible subjects were assigned to receive either a single dose of DAB₃₈₉IL-2 (18 μg/kg) followed by vaccination with tumor RNA-transfected DC, or to vaccination alone. All subjects received 3 intradermal injections of tumor RNA-transfected DC. The injections were given intradermally at biweekly intervals and consisted of 1×10⁷ cells suspended in 200 μL 0.9% sodium chloride at each vaccination. Following treatment, subjects were evaluated for clinical toxicity, immunological and clinical responses. Due to regulatory restrictions and, in some subjects, limited access to tumor tissue, no tumor biopsies were performed.

DAB₃₈₉IL-2 and Vaccine Preparation

DAB₃₈₉IL-2 (ONTAK™, Ligand Pharmaceuticals) was provided as a frozen, sterile solution formulated in citrate buffer in 2 ml single use vials at a concentration of 150 μg/ml. After thawing, DAB₃₈₉IL-2 was diluted with sterile normal saline to a final concentration of 15 μg/ml and delivered by intravenous infusion over a 30-minute period. Patients were permitted to receive acetaminophen (600 mg) and antihistamines 30 to 60 minutes prior to infusion. For DC culture, a concentrated leukocyte fraction was harvested by leukapheresis. PBMC were isolated from the leukapheresis product by density gradient centrifugation (HISTOPAQUE®, Sigma). The semi-adherent cell fraction was used for DC culture in serum-free X-VIVO 15™ medium (Cambrex Bio Science) supplemented with rhIL-4 (500 U/ml) (R&D Systems) and rhGM-CSF (800 U/ml) (Immunex). After 7 days, immature DC were harvested and transfected with total RNA extracted from tumor tissues histologically classified as clear cell carcinoma. Control RNA used for immunological monitoring studies was isolated from autologous benign renal tissues (RE) or from PBMC. Transfection of immature DC was carried out by electroporation. DC were washed in PBS and resuspended at a concentration of 4×10⁷ cells/ml in ViaSpan® (Barr Laboratories). Cells were then coincubated for 5 minutes with 5 μg RNA per 1×10⁶ cells and electroporated in 0.4 cm cuvettes via exponential decay delivery at 300V and 150° F. (Gene Pulser II, BioRad). After electroporation, cells were resuspended in X-VIVO 15™ medium, and matured for 20 hours in the presence of 10 ng/ml TNF-α, 10 ng/ml IL-1β, 150 ng/ml IL-6 (R&D Systems), and 1 μg/ml PGE₂ (Cayman Chemicals). Prior to administration, cells were characterized to ensure that they met the typical phenotype of fully mature DCs: Lin^{neg}, HLA class I and II^{high}, CD86^{high}, CD83^{high}.

Evaluation of Immune Status

Interferon-γ and IL-4 ELISPOT analyses were performed using PBMC obtained prior to, during, and after vaccination. PBMC were cultured overnight in complete RPMI 1640 medium. CD4⁺ and CD8⁺ T cells were isolated from PBMC by negative depletion (Miltenyi). After blocking, 1×10⁵ T cells and 1×10⁴ RNA-transfected DC were added to each well of 96-well nitrocellulose plates (Multiscreen-IP, Millipore) precoated with 2 μg/ml Interferon-γ capture antibody (Endogen) or with IL-4 capture antibody (BD Biosciences Pharmingen). Plates were incubated for 20 hours at 37° C., and biotinylated Interferon-γ detection antibody (Endogen) or biotinylated IL-4 antibody (BD Biosciences Pharmingen) was added to each well. Cells were then incubated for an additional two hours at room temperature, then with streptavidin-alkaline phosphatase (1 μg/ml; Sigma) and plates were developed with substrate (Kirkegaard & Perry Laboratories). After washing, spots were counted using an automated ELISPOT reader (Carl Zeiss). CTL assays were performed by coculturing RNA-transfected DC with autologous PBMC. Cells were restimulated once and IL-2 (20 units/ml) was added after five days and every other day thereafter. After 12 days of culture, effector cells were harvested. Target cells were labeled with 100 μCi of Na₂[⁵¹CrO₄] (NEN) in 200 μl of complete RPMI 1640 for one hour at 37° C. in 5% CO₂ and ⁵¹Cr-labeled target cells were incubated in complete RPMI 1640 medium with effector cells for five hours at 37° C. Then, 50 μl of supernatant was harvested, and release of ⁵¹Cr was measured with a scintillation counter. For proliferation assays, purified CD3⁺ T cells were seeded into round-bottomed microplates in the presence of mRNA-transfected DC. T cells alone were used as the background control. After 4 days, 1 μCi of [methyl-³H] thymidine (NEN) was added to each well for an additional 16 hours. Incorporation of thymidine was determined using a liquid scintillation counter. Cytotoxicity of DAB₃₈₉IL-2 was determined in MTT (3-(4,5-dimethylthazol-2-yl)-2,5-diphenyl tetrazolium bromide salt) assays. After a 6-hour incubation with varying concentrations of DAB₃₈₉IL-2, cells were seeded in 96-well plates at a density of 5×10³ cells/well. After 48 hours of incubation, 20 μL MTT from a 5 mg/mL stock was added. After 4 hours, the formazan crystals were solubilized by adding 100 μL isopropanol/0.1 M hydrochloric acid. The absorbance of the formazan product was measured on an ELISA plate reader at 570 nm.

Fluorescence-Activated Cell Sorter (FACS) Analysis

Four-color FACS analyses were performed using the following antibodies: anti-CD4 FITC, anti-CD45RO, anti-CD45RA (Caltag), anti-CD25 PE (Becton Dickinson), anti-GITR (R&D Systems), and isotypic controls (Caltag). Sorting of CD4⁺/CD25^{neg}, CD4⁺/CD25^{int} and CD4⁺/CD25^{high} T cells was performed using a Becton Dickinson FACSAria™ cell sorter after antibody labeling. For intracellular detection of FoxP3, cells were permeabilized with 30 μg/ml digitonin for 45 minutes at 4° C. Subsequently, cells were stained with anti-FoxP3 antibody (Abcam), and R-PE anti-goat IgG in the presence of 10 μg/ml digitonin for 30 minutes at 4° C. Following staining, cells were fixed and analyzed by FACS. For intracellular CTLA-4 detection, T cells were permeabilized, fixed, and stained with biotinylated anti-CD152 (Becton Dickinson) followed by APC-streptavidin (Becton Dickinson). A total of 1×10⁶ cells was suspended in staining buffer (PBS with 1% FCS, 2 mM EDTA, and 0.1% sodium azide) and incubated for 20 minutes at 4° C. with the antibody.

T_{reg} Functional Evaluation

The suppressive activity of T_{reg} isolated from PBMC of study subjects prior to and 4 days after DAB₃₈₆IL-2 administration was analyzed, as described previously²⁵. CD4⁺/CD25⁺ T cells were isolated from the PBMC of study subjects using magnetic bead separation techniques. Cells were washed with PBS, resuspended in complete RPMI 1640 medium, and placed into 96-well round bottom plates pre-coated with anti-CD3/CD28 antibodies (0.4 μg/well) (Caltag). CD4⁺/CD25⁻ cells were plated at 2.0×10⁴/well alone or in combination with CD4⁺/CD25⁺ cells in triplicate wells at a ratio of 1:2 (CD4⁺/CD25⁻:CD4⁺CD25⁺). On day 5, 1 μCi of ³H thymidine was added for the final 16 hr of the cultures. Cells were then harvested on glass fiber filters and assessed for uptake of radiolabeled thymidine.

Detection of FoxP3 Transcripts

Details of real-time PCR-based quantification of FoxP3 transcripts were previously provided by Heiser et al.¹ FoxP3 forward primer 5′-TCCCAGAGTTCCTCCACAAC-3′ (SEQ ID NO:1), reverse primer 5′-ATTGAGTGTCCGCTGCTTCT-3′ (SEQ ID NO:2), and the fluorogenic probe 5′-FAM-CTACGCCACGCTCATCCGCT-TAMRA-3′ (SEQ ID NO:3) were used at a concentration of 250 nM.

Statistical Analysis

Pre- and post-treatment T-cell analysis was performed by Interferon-γ ELISPOT on all patients who completed immunotherapy. Increases of antigen-specific CD4⁺ and CD8⁺ T cells after vaccination were compared using the Wilcoxon matched-pairs signed rank test, analyzing the null hypothesis that the rates of change in T-cell response were equivalent prior to and after therapy. A two-sided p-value of <0.05 was considered statistically significant.

Phenotypic and Functional Characterization of Regulatory T Cells

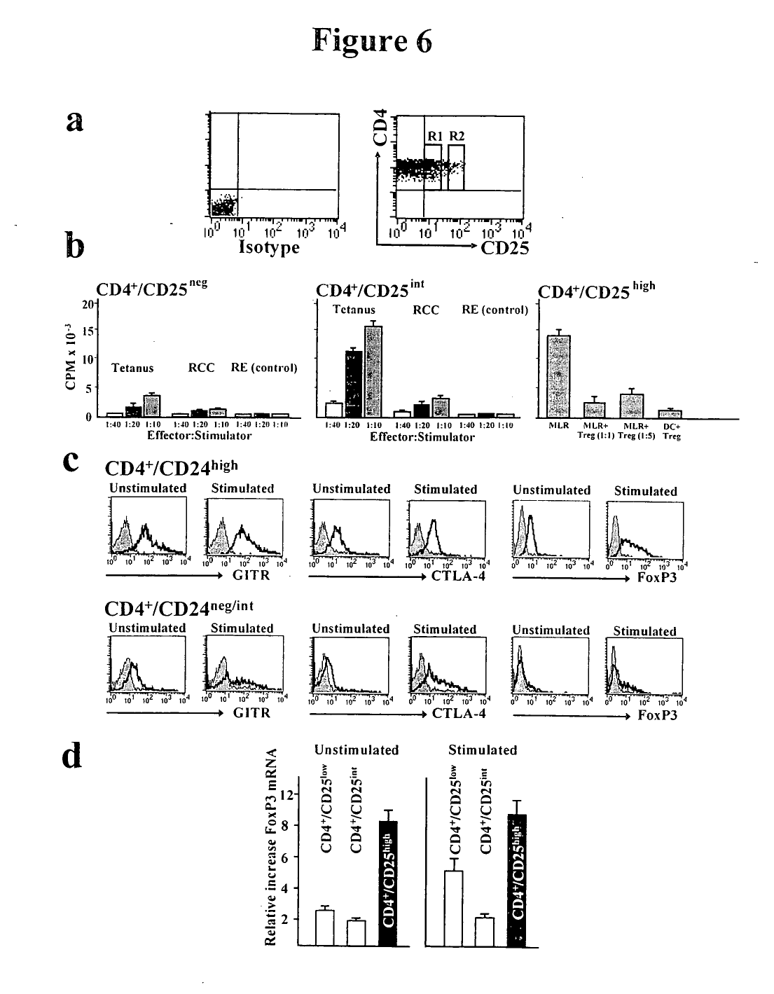

Human CD4⁺ T cells expressing CD25 represent a heterogeneous cell population containing not only regulatory, but also effector/memory T cells¹³. Analysis of PBMC from healthy donors and RCC patients revealed the presence of CD4⁺ T-cell populations that express increasing levels of CD25¹³. As shown in FIG. 6A, one major subset of CD4⁺ T cells, isolated from the PBMC of a RCC patient, lacked CD25 expression, while a second population was characterized by intermediate levels of CD25 (R1), and a third, albeit small portion, exhibited high CD25 cell surface expression levels (R2). To further characterize these 3 subsets, CD4⁺/CD25^{neg}, CD4⁺/CD25^{int}, and CD4⁺/CD25^{high} T cells were isolated from the PBMC of RCC patients by FACS and were functionally analyzed in vitro (FIG. 6B). CD4⁺/CD25^{neg} cells expressed cell surface markers characteristic of naïve/resting T cells and demonstrated reduced proliferative responses following exposure to tetanus toxoid (Tetanus), renal tumor RNA-(RCC), benign renal epithelium RNA-(RE), and PBMC RNA-loaded dendritic cells (DC). In contrast, CD4⁺/CD25^{int} cells produced a strong proliferative response against tetanus toxoid, and a significant, albeit weaker response, against RCC RNA-encoded antigens. No proliferative response against RE RNA- or PBMC RNA-transfected DC was observed.

CD4⁺/CD25^{high} T_{reg} exhibited profound immunosuppressive activity in vitro, as evidenced by inhibition of allogeneic DC-stimulated mixed lymphocyte reaction (MLR) cultures. The addition of increasing numbers of CD4⁺/CD25^{high} cells (1/5 responder cells; 1/1 responder cells) to MLR reactions led to a dose-dependent inhibition of responder T-cell proliferation, while CD4⁺/CD25^{high} T cells did not proliferate significantly upon stimulation with DC (DC+T_{reg}).

T_{reg} demonstrated strong cell surface expression of GITR as well as intracellular CTLA-4 and FoxP3 (FIG. 6C). Stimulation of CD4⁺/CD25^{high} T cells using anti-CD3/CD28 antibodies resulted in enhanced expression of GITR, CTLA-4, and FoxP3, while CD4⁺ T cells with negative or intermediate levels of CD25 displayed significantly lower levels of these markers after unspecific stimulation. Consistent with other reports^{6,14}, quantitative real-time PCR confirmed high expression of FoxP3 transcripts by T_{reg} when compared to CD4⁺/CD25^{neg} or CD4⁺/CD25^{int} T-cell subsets (FIG. 6D). Following unspecific T-cell receptor stimulation, FoxP3 mRNA copy numbers were found to be significantly increased in CD4⁺/CD25^{neg} subsets, while CD4⁺/CD25^{int} and CD4⁺/CD25^{high} T-cell populations demonstrated only modest FoxP3 upregulation (FIG. 6D). In this study, T_{reg} frequencies detected in the peripheral blood of metastatic RCC patients after tumor nephrectomy (n=10) ranged from 2.5% to 4.6% and were elevated (2.1±1.2-fold increase), when compared to healthy volunteer controls (n=4).

CD4⁺/CD25^{high} T cells isolated from the PBMC of RCC patients exhibit suppressive activity, while CD4⁺ cells with negative or intermediate CD25 levels represent either naïve/resting or memory/effector T cells. Therefore, in clinical settings, it will be important to identify suitable reagents that allow selective elimination of CD25^{high} T_{reg}, while sparing other cells expressing low or intermediate levels of CD25. Consistent with other reports^{6,15}, higher T_{reg} frequencies were measured in the peripheral blood of metastatic RCC patients, when compared to healthy donor controls.

Selective Elimination of Regulatory T Cells In Vitro

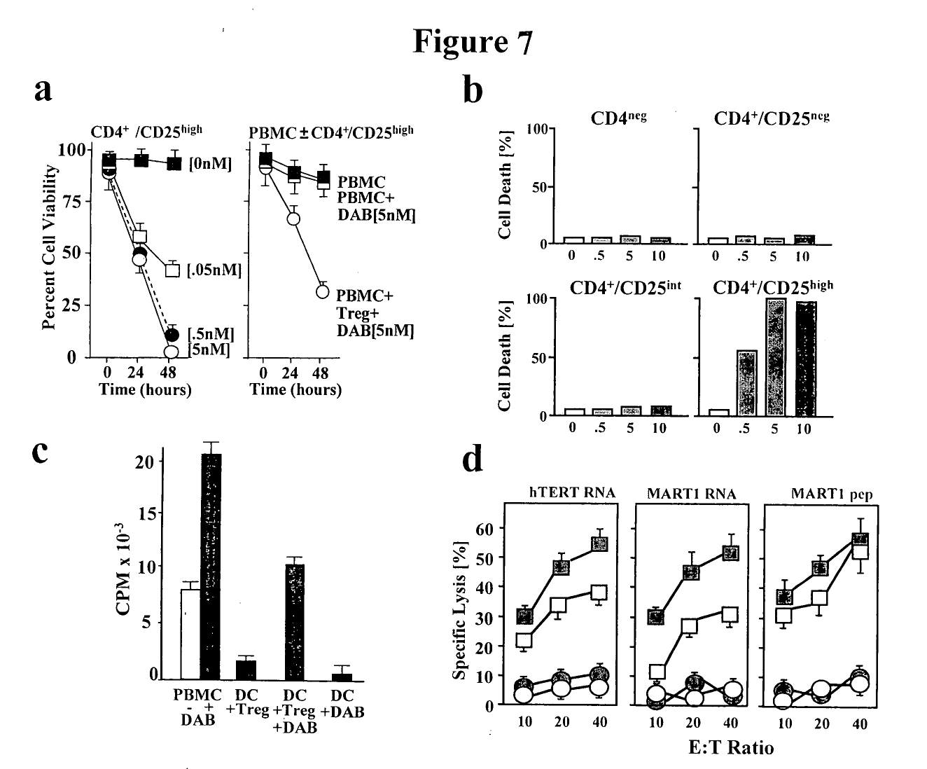

Human malignant cells overexpressing CD25 can be inactivated or eliminated using the recombinant IL-2 diphtheria toxin conjugate, denileukin difititox (DAB₃₈₉IL-2)¹⁶. In order to determine whether DAB₃₈₉IL-2 could serve as a suitable reagent to achieve T_{reg} depletion under clinically relevant conditions, T_{reg} susceptibility to DAB₃₈₉IL-2 was analyzed in MTT assays. In these experiments, conditions were chosen that resembled the pharmacokinetics of a single intravenous dose of DAB₃₈₉IL-2 (18 μg/kg) corresponding to 5 nM peak plasma concentrations. Given a plasma half-life of approximately 60 minutes and a dissociation constant of 1 pM for DAB₃₈₉IL-2 and the high-affinity IL-2 receptor, DAB₃₈₉IL-2 plasma levels were projected to reach suboptimal concentrations after 6 hours. Therefore, in the experiments shown in FIG. 7A, the viability of isolated CD4⁺/CD25^{high} T cells was analyzed after a 6-hour exposure to increasing concentrations of DAB₃₈₉IL-2 (range 0.05-5.0 nM) in vitro over 48 hours. For CD4⁺/CD25^{high} T_{reg}, a significant reduction in cell viability was observed 24 hours following exposure with DAB₃₈₉IL-2. Efficient killing of CD4⁺/CD25^{high} cells was noted at 0.5 nM concentrations after 48 hours, while complete depletion was achieved at a 5 nM concentration. In contrast, exposure of CD4⁺/CD25^{neg} and CD4⁺/CD25^{int} cells to DAB₃₈₉IL-2 did not result in significant cell death, except when these cells were exposed to DAB₃₈₉IL-2 concentrations higher than 10 nM. In another set of experiments, DAB₃₈₉IL-2, used at a 5 nM concentration, resulted in specific killing of T_{reg}, but not of other bystander cells in vitro.

As shown in FIG. 7A, there were no significant differences in PBMC viability over time when DAB₃₈₉IL-2 [5 nM] was added to the culture. In contrast, when PBMC and T_{reg} were mixed (1:1 ratio), cell viability was reduced by more than 60% after 48 hours, indicating selective, diphtheria toxin-mediated-T_{reg} killing without bystander toxicity. In order to corroborate these findings, 7-AAD staining was performed on CD4⁺ T-cell populations with increasing densities of CD25 (FIG. 7B). Consistent with the experiments shown in FIG. 7A, optimal killing of CD4⁺/CD25^{high} T_{reg} was achieved using 5 nM DAB₃₈₉IL-2 concentrations, while DAB₃₈₉IL-2 exposure to other CD4⁺ T cells with lower or no CD25 expression revealed only background levels of 7-AAD staining.

Next, the impact of DAB₃₈₉IL-2 on freshly activated lymphocytes was analyzed after stimulation with allogeneic DC in MLR cultures. DAB₃₈₉IL-2-mediated T_{reg} depletion prior to initiation of the MLR culture resulted in a two-fold increase in proliferation of responder cells (PBMC±DAB). Conversely, the addition of isolated T_{reg} (DC+T_{reg}) resulted in an approximately 80% reduction of T-cell proliferation (1:1 T_{reg} to responder ratio) (FIG. 7C). Pre-incubation of T_{reg} with 5 nM DAB₃₈₉IL-2 (DC+T_{reg}+DAB) significantly abrogated their inhibitory effect when added to the MLR reaction, however, responder cells did not proliferate as vigorously as in the absence of T_{reg}, indicating potential contact inhibition by T_{reg}, as reported previously¹⁷. Importantly, the addition of DAB₃₈₉IL-2 two days after initiation of the MLR reaction (DC+DAB) completely abrogated the proliferation of responder cells, indicating that DAB₃₈₉IL-2 eliminates not only T_{reg}, but also freshly activated naïve T cells that acquire CD25 expression.

These experiments show that DAB₃₈₉IL-2 is a suitable reagent for selectively eliminating T_{reg} in vitro without affecting other lymphocytes, including naïve and memory T cells with negative or intermediate expression levels of CD25, respectively. These data further indicate that, in a vaccination setting, DAB₃₈₉IL-2 should only be applied prior to immunization, but not during vaccination phase, since activated effector T cells appear susceptible to DAB₃₈₉IL-2-mediated toxicity.

Enhancement of T-Cell Responses after Regulatory T-Cell Depletion In Vitro

To provide evidence that DAB₃₈₉IL-2-mediated T_{reg} depletion is capable of augmenting antigen-specific T-cell responses in vitro, CTL were stimulated from PBMC that were pretreated with or without DAB₃₈₉IL-2 [5 nM] (FIG. 7D). PBMC were stimulated twice with autologous DC transfected with human telomerase reverse transcriptase-(hTERT) and MART-1 mRNA. In addition, DC pulsed with an HLA-A0201-restricted MART-1 peptide were used as stimulators. RNA-transfected DC were not only used as stimulators, but also served as specific or control targets, as described previously^{1,18-20}. The ability of the stimulated, antigen-specific CTL to recognize their cognate, but not control target cells, was analyzed in standard cytotoxicity assays. As shown in FIG. 7D, CTL stimulated from T_{reg}-depleted PBMC exhibited significantly higher lytic activity against antigens encoded by hTERT- or MART-1 mRNA than CTL stimulated from non-depleted PBMC. In contrast, only modest, statistically insignificant improvement of antigen-specific killing was observed when MART-1 peptide-pulsed DC were used as stimulators.

These data show that depletion of T_{reg} from human PBMC enhances the stimulation of antigen-specific CTL in vitro. It appears, that among other factors, the impact of this strategy is influenced by the strength of the antigenic signal²¹, as evidenced by the fact that T-cell responses against mRNA-encoded self-antigens were significantly enhanced. In contrast, the T_{reg} depletion strategy was less effective in improving CTL responses, when DC presenting high densities of peptide-MHC complexes (peptide pulsing) were used for stimulation.

Depletion of Regulatory T Cells In Vivo

Having shown that T_{reg} elimination is capable of enhancing T-cell responses in vitro, a clinical study was initiated to test the T_{reg} depletion concept in a human vaccination setting. Eleven patients including 10 with metastatic RCC and one with disseminated ovarian carcinoma (OVA) were treated in an Institutional Review Board (IRB) and Food and Drug Administration (FDA)-approved study. Seven subjects received a single intravenous dose of DAB₃₈₉IL-2 (18 μg/kg), 4 days prior to vaccination with tumor RNA-transfected DC, while a second cohort of 4 subjects was treated with the vaccine alone (Table 2). Outside the study, PBMC were obtained from one additional RCC subject who received a single dose of DAB₃₈₉IL-2 (18 μg/kg), but no vaccine. Consistent with reports of others, DAB₃₈₉IL-2-related toxicities included grade I constitutional symptoms such as low-grade fever and malaise (n=4), grade I elevation of serum rheumatoid factor (n=1), and transient grade II serum alanine aminotransferase (ALT) elevations (n=1)²². RNA-transfected DC injections were well tolerated without any major clinical toxicities or serologic/immunologic evidence of autoimmunity^{1,2.}

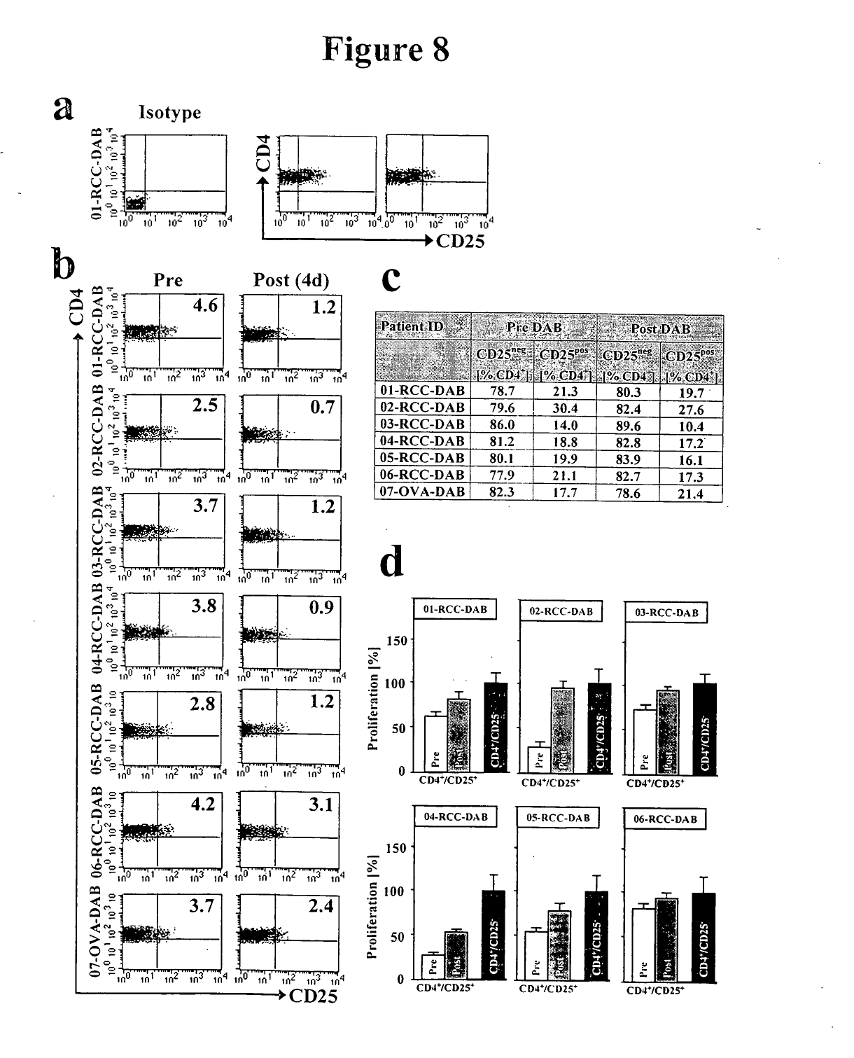

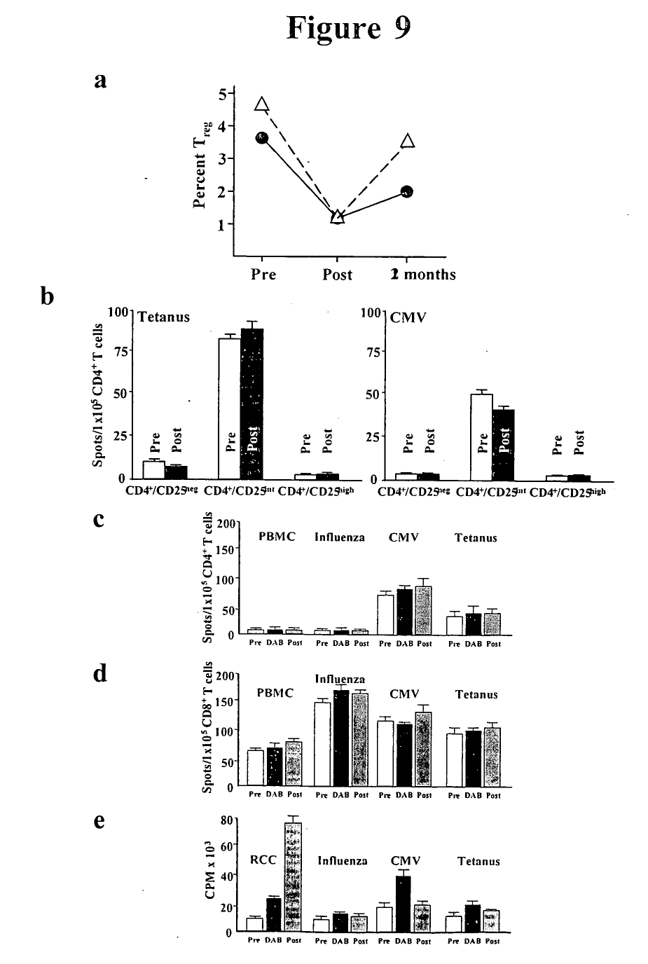

In order to quantify the presence of CD4⁺/CD25^{neg}, CD4⁺/CD25^{int}, and CD4⁺/CD25^{high} T cells in PBMC samples collected prior to and after DAB₃₈₉IL-2 treatment (FIG. 8A), flow cytometry was performed on patient-derived samples using identical settings (gates) as shown in FIG. 6A. As demonstrated in FIG. 8B, DAB₃₈₉IL-2 administration resulted in a significant reduction (range 26% to 76%) of CD4⁺/CD25^{high} T_{reg} in all 7 patients, at 4 days following intravenous infusion. Additional evidence indicating T_{reg} depletion was provided by the observation that the number of total CD25^{pos} cells measured in each subject after DAB₃₈₉IL-2 administration decreased correspondingly with the number of depleted CD4⁺/CD25^{high} T_{reg}, providing evidence that CD25^{neg/int} subsets were unaffected (FIG. 8C). Finally, CD4⁺/CD25⁺ T cells isolated prior (Pre), but not 4 days after (Post) DAB₃₈₉IL-2 treatment, consistently inhibited anti-CD3/CD28-mediated activation of CD4⁺/CD25⁻ indicator T cells in all subjects analyzed (FIG. 8D), indicating abrogation of T_{reg}-mediated immunosuppressive activity in vivo. Notably, DAB₃₈₉IL-2-mediated T_{reg} elimination was transient, since approximately 75% of T_{reg} were restored within two months in the patients' peripheral T-cell pool (FIG. 9A).

In 5 of 6 evaluable DAB₃₈₉IL-2-treated subjects, minor reductions (averaging 10%) in absolute neutrophil counts (ANC) were observed, while one subject exhibited a decrease of 20%. Accordingly, among these 5 with only minor ANC reductions, there were no significant changes in the relative number of CD3⁺, CD4⁺, CD8⁺ T cells, B cells, monocytes/macrophages (CD14), and NK cells after treatment.

In order to address the concern that DAB₃₈₉IL-2-mediated T_{reg} depletion may induce toxicity within the memory T-cell pool (CD4⁺/CD25^{int}), a series of experiments was performed analyzing CD4⁺/CD25^{int} T-cell function prior to (Pre) and 4 days (Post) after DAB₃₈₉IL-2 administration. First, interferon-γ ELISPOT analyses were performed on sorted CD4⁺/CD25^{neg}, CD4⁺/CD25^{int}, and CD4⁺/CD25^{high} T-cell subsets using tetanus toxoid (Tetanus) or cytomegalovirus (CMV) lysate-pulsed DC as stimulators. As shown in FIG. 9B, only CD4⁺/CD25^{int} memory T cells were capable of stimulating T-cell responses against tetanus or CMV antigens, while naïve (CD4⁺/CD25^{neg}) and CD4⁺/CD25^{high} T cells failed to stimulate T-cell responses of a significant magnitude. In a second set of experiments, the frequency of Interferon-γ secreting T cells was analyzed using CD4⁺ (FIG. 9C) and CD8⁺ responder T cells isolated from human PBMC (FIG. 9D) prior to (Pre), 4 days after DAB₃₈₉IL-2 administration (DAB), and 2 weeks after 3 vaccination cycles (Post). Over the entire treatment cycle, no differences were observed in the absolute number of T-cells with specificity against PBMC RNA-transfected DC, fluM1 mRNA-transfected DC (influenza), CMV lysate-loaded DC, or tetanus toxoid-loaded DC. Furthermore, antigen-specific proliferation assays (FIG. 9E) revealed strong reactivities against renal tumor antigens (RCC RNA-transfected DC), and unchanged reactivities against the prototype recall antigens fluM1 (influenza)/tetanus toxoid (Tetanus), and against CMV.

Cumulatively, the experiments shown in FIGS. 8 and 9 demonstrate that administration of a single dose of DAB₃₈₉IL-2 resulted in significantly reduced numbers of T_{reg} in the peripheral blood of RCC patients (FIG. 8B), and in significant abrogation of T_{reg}-mediated suppressive activity (FIG. 8D). These data further indicate that DAB₃₈₉IL-2-mediated toxicities against other hematopoietic cells expressing CD25 are unlikely, and that lymphopenia-induced T-cell proliferation²³ may not represent a significant issue in a vaccination setting.

In Vivo Stimulation of Tumor-Specific T-Cell Responses

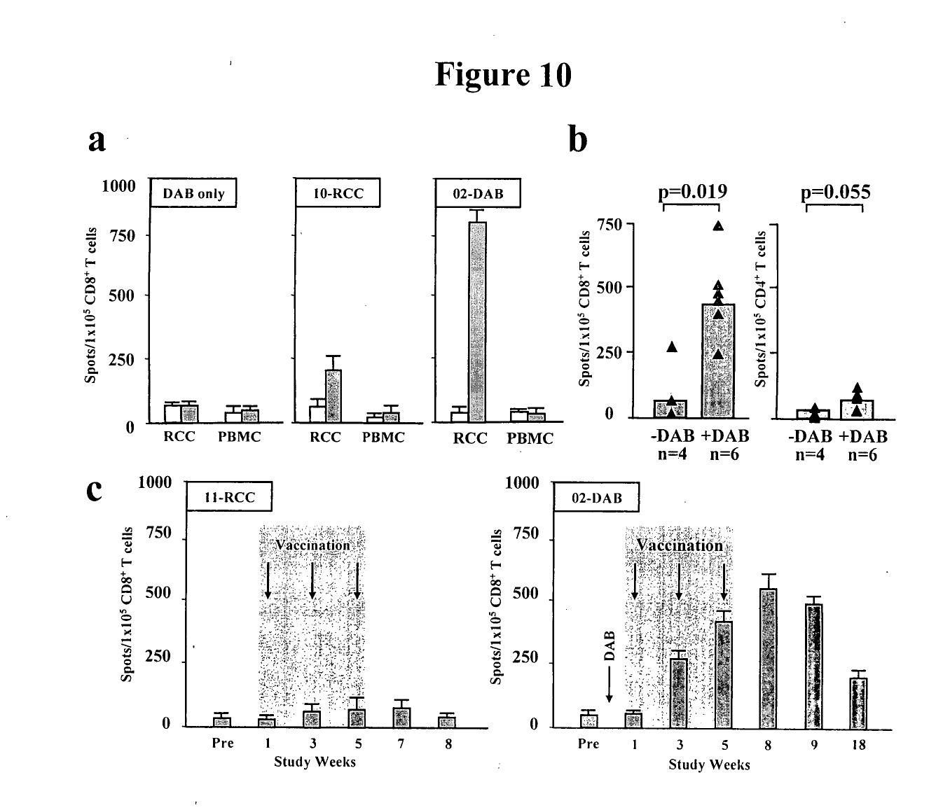

In order to determine whether DAB₃₈₉IL-2-mediated T_{reg} depletion is capable of augmenting vaccine-induced CD8⁺ and CD4⁺ T-cell responses in cancer patients, Interferon-γ ELISPOT analyses were performed to determine the frequencies of vaccine-induced, tumor-specific T cells from PBMC samples collected before and two weeks after the third vaccination. CD8⁺ and CD4⁺ T cells were isolated from pre- and post-vaccination PBMC and cultured overnight with tumor RNA-transfected DC targets. As controls, autologous PBMC RNA or autologous benign renal epithelium (RE)-derived RNA-transfected DC were used for short-term antigenic stimulation. As exemplified by the 3 study subjects shown in FIG. 10A, only background levels of PBMC-specific T cells were observed before (white bars) or after vaccination (grey bars). Also, tumor-specific T-cell responses did not significantly increase after treatment with DAB₃₈₉IL-2 alone over a period of 28 days (exemplified in subject DAB only). In contrast, all patients immunized with tumor RNA-transfected DC exhibited significant increases in tumor-specific CD8⁺ and CD4⁺ T-cell frequencies after vaccination (Table 2). A 2.7-fold median increase (range 0.3 to 5.1) of tumor-specific CD8⁺ T cells, and a 2.0-fold median increase in tumor-specific CD4⁺ T cells (range 1.0 to 4.5) was observed in the subjects receiving vaccination alone (exemplified by subject 10-RCC, FIG. 10A).

Although there was significant patient-to-patient variability in the magnitude of T-cell responses measured in each patient, vaccination after T_{reg} depletion stimulated significantly higher numbers of tumor-specific CD8⁺ T cells in RCC patients receiving DAB₃₈₉IL-2 plus RCC RNA-transfected DC, when compared to RCC patients subjects receiving vaccination alone (p=0.014). Moreover, there was a trend towards improved CD4⁺ T-cell responses (p=0.055) in RCC patients treated with combined therapy (FIG. 10B). A 7.9-fold median increase in the number of tumor-specific CD8⁺ T cells (range 5.4 to 16.0), and a 7.2-fold median increase in CD4⁺ T cells (range 2.2 to 31.0), was detected in the 6 RCC patients receiving DAB₃₈₉IL-2 plus vaccination with RCC RNA-transfected DC. The absolute CD8⁺ T-cell frequencies achieved with combined therapy were remarkably high with up to 0.81% of CD8⁺ T cells exhibiting tumor specificity after 3 vaccinations. In order to evaluate the temporal evolution of the T-cell response stimulated by vaccination with or without DAB₃₈₆IL-2 therapy, longitudinal monitoring of vaccine-induced CD8⁺ T-cell responses was performed using Interferon-γ ELISPOT in 2 subjects from whom sufficient numbers of cells were available for analysis (11-RCC; 02-DAB). DAB₃₈₉IL-2-mediated T_{reg} depletion followed by vaccination resulted in a significantly enhanced and also prolonged CD8⁺ T-cell response, when compared to the subject receiving vaccination alone. The vaccine-induced and tumor-specific CTL response surged over the entire treatment course and peaked approximately 2 weeks after the 3^{rd} and final dose (FIG. 10C).

T_{reg} depletion using the diphtheria fusion protein DAB₃₈₉IL-2 is capable of enhancing a vaccine-induced T-cell response in advanced RCC patients. Although only a limited number of patients were studied in this clinical trial, an up to 16-fold increase in tumor-specific CTL frequencies could be measured in subjects receiving combined treatment, when compared to individuals receiving vaccination alone. The vaccine-induced T-cell frequencies achieved without T_{reg} depletion were similar to those observed in a prior study in which immature tumor RNA-transfected DC were used for vaccination²⁴.

Although in these examples, the concept of regulatory T cell elimination has been reduced to practice in context with RNA transfected DC-based vaccination, it is readily recognized that, on the basis of the discovery of the present invention, this strategy could be applied to any immune-based approach including active and passive immunotherapy, as well as to classical adjuvants.

### Example 3

Presence and Phenotype of ImC in Patients with Metastatic RCC after Nephrectomy.

Initial experiments were conducted to determine whether ImC with suppressive activity can be isolated from PBMC of patients with metastatic RCC (after tumor nephrectomy) and of healthy volunteers. PBMC were isolated from whole blood and T cells (CD3) were depleted via magnetic bead separation techniques. Cells were resuspended in RPMI 1640 supplemented with human AB serum and 30 ng/ml of GM-CSF to sustain cell viability. Cells were incubated at 37° C./5% CO₂ for 24-48 hours. Non-adherent cells were harvested and monocytic precursors were isolated using OptiPrep™ density gradient medium and labeled with PE-conjugated, lineage-specific (CD3, CD14, CD19, CD56) antibodies and FITC-conjugated HLA-DR antibody. ImC were isolated by FACS sorting of Lin⁻/HLA-DR⁻ cell populations. For phenotypic analysis, sorted ImC populations were labeled with antibodies directed against CD1a (DC marker), CD10 (lymphoid marker), CD11b (myeloid marker), CD13 (aminopeptidase N), CD15 (Lewis X Antigen), CD18 (ICAM-1), CD31, CD33 (myeloid cell markers), HLA ABC, and HLA-DR. Healthy volunteers (n=10) exhibited only low numbers of Lin⁻/DR⁻ ImC, whereas patients with advanced-stage RCC (n=12) generally exhibited higher frequencies of ImC in their peripheral blood (5.7±2.6-fold increase). Cytopathologic analysis of Lin⁻/DR⁻ ImC revealed cell morphology consistent with cells of myeloid origin exhibiting typical cytoplasmic granulations. The isolated Lin⁻/HLA-DR⁻ ImC exhibited high levels of HLA class I, CD18, CD33, and intermediate cell surface expression for CD1a, CD10, CD13, CD31, and CD11b.

These data show that Lin⁻/HLA-DR⁻ ImC are considerably elevated in the peripheral blood of metastatic RCC patients (despite tumor nephrectomy), when compared to healthy volunteers.

Functional Validation of Lin⁻/HLA-DR⁻ ImC.

In order to functionally characterize Lin⁻/HLA-DR⁻ ImC, their capability of inhibiting an antigen-specific T-cell response in vitro was evaluated. An analysis was done to determine whether the addition of tetanus toxoid (TT)-loaded Lin⁻/HLA-DR⁻ImC could inhibit T-cell proliferation against the model antigen TT. T cells were isolated from the PBMC of a RCC patient and then stimulated with autologous, TT-loaded mature DC (DC). TT-loaded Lin⁻/HLA-DR⁻ ImC or Lin⁻/HLA-DR⁺ (control) cells were added to cultures at a 1:1 stimulator/ImC ratio. As controls, unloaded (DC) or TT-loaded DC (DC+TT) were used to determine antigen-specific proliferation by measuring [³H]thymidine incorporation. The addition of TT-loaded ImC significantly inhibited TT-specific T-cell proliferation, while TT-loaded Lin⁻/HLA-DR⁺ cells (immature DC-like cells) enhanced a DC-mediated proliferative response against TT. Unloaded DC stimulated only background levels of T-cell proliferation.

In order to provide evidence that Lin⁻/HLA-DR⁻ ImC are also capable of inhibiting antigen-specific CD8⁺₋T-cell responses, their immunosuppressive activity was tested in standard cytotoxicity or Interferon-γ ELISPOT assays, using ImC from a HLA-A2⁺ RCC patient. For CTL assays, HLA-A201-restricted MART-1-specific CTL (MART-1 CTL clone) were cultured for 5 h with ⁵¹Cr-labeled, MART-1 peptide-loaded T2 target cells at a 1:1 ratio. Identical numbers of MART-1 peptide-(RCC Lin⁻/DR⁻/MART-1), or control (RCC Lin⁻/DR⁻/Control) peptide-loaded ImC (Lin⁻/HLA-DR⁻), isolated from the same RCC patient, were then added to individual effector/target cell aliquots. As controls, peptide-loaded T2 cells (Cold Target), or ImC isolated from the PBMC of a HLA-A2⁺ healthy donor (Donor) were added to individual cultures. MART-1 peptide-loaded ImC significantly inhibited CTL-mediated lysis or Interferon-γ secretion in an antigen-specific fashion, while control peptide-loaded ImC, MART-1 peptide-loaded healthy donor ImC, and unloaded T2 cells exhibited no or only modest inhibitory activity.

Lin⁻/HLA-DR⁻ ImC Express Nuclear Retinoic Acid Receptors.

Studies were carried out to determine if Lin⁻/HLA-DR⁻ImC, Lin⁻/HLA-DR⁺ cells, and HL-60 leukemic cells (control) express the retinoic acid receptor (RAR) isoforms α, β, and γ. Experiments were also conducted to screen for ImC-mediated expression of selected cytokines and enzymes implicated in mediating immune suppression. For these experiments, ImC were generated as described and RT PCR was performed using primers specific for RAR α, β, γ, iNOS, COX-2, G-CSF, TNF-α, and CD115. From all cell populations tested, RAR α, β, and γ transcripts could be amplified. Moreover, high levels of CD115, iNOS, G-CSF and TNF-α, and COX-2 transcripts could consistently be amplified from Lin⁻/HLA-DR⁻ ImC and control HL-60 cells.

In summary, these studies show that Lin⁻/HLA-DR⁻ ImC express nucleic retinoic acid receptors and also express enzymes and cytokines promoting the development, survival, and immunosuppressive function of ImC.

Isolation of ImC Using the Myeloid Marker CD33.

Studies were conducted to determine if ImC populations can be isolated and characterized based on positive selection for CD33. In these experiments, the frequencies of CD33⁺/HLA-DR⁻/Lin⁻ cells present in the PBMC of a healthy donor and in 3 metastatic RCC patients was demonstrated. PBMC were gated on Lin⁻/HLA-DR⁻ and analyzed for expression of CD33. Lin⁻/HLA-DR⁻/CD33⁺ ImC were significantly increased in the RCC patients (1.92; 1.44; 0.98%) when compared to the healthy volunteer (0.42%). It was also shown by forward and side scatter plotting that the isolated CD33⁺/HLA-DR⁻ ImC represent a homogeneous and distinct myeloid cell population within the total PBMC pool.

These data demonstrate that ImC can be isolated from subjects using the myeloid marker CD33. Consistent with the data above, CD33⁺/HLA-DR⁻ ImC represent a homogeneous cell population that is significantly elevated in RCC patients when compared to healthy volunteers.

Isolation and Phenotype of CD33⁺ ImC.

Cell surface expression of CD33 is only present within the Lineage-negative and HLA-DR-positive cell population, but not within the Lineage-positive and HLA-DR-negative cell population (predominantly T cells and NK cells). Accordingly, the isolation of ImC from PBMC can be greatly simplified by isolating ImC via CD33 positive selection of HLA-DR and CD15 (granulocyte)-depleted cells. Separation of PBMC using HLA-DR and CD15 magnetic beads leads to a selective depletion of granulocytes, monocytes, macrophages, and B cells, respectively. Subsequent positive selection with anti-CD33 results in depletion of predominantly T cells and NK cells and yields a homogeneous cell population exhibiting high expression of HLA class I and M-CSF (CD115).

Acquisition of HLA Class II and Co-Stimulatory Molecule Expression after ATRA Treatment.

The isolated CD33⁺/DR⁻ ImC were further characterized by phenotypic and functional analyses. Experiments were also conducted to determine whether the phenotype and function of CD33⁺ ImC can be modulated in vitro by the differentiation agent all-trans retinoic acid (ATRA (Tretinoin)).

For phenotypic analysis, CD33⁺/DR⁻ ImC isolated as described were cultured for 4 days in GM-CSF-containing medium in the absence and in the presence of ATRA [1 μM]. CD33⁺/HLA-DR⁻ ImC exhibited a phenotype identical to Lin⁻/HLA-DR⁻ ImC: CD33^{high}, CD11c^{high}, HLA class I^{high}, CD1a^{int}, HLA-DR^{neg}, CD40^{neg}, and CD86^{neg}. In vitro exposure to ATRA [1 μM] resulted in differentiation of ImC, as evidenced by acquisition of the cell surface markers HLA-DR, CD40, and CD86.

In order to provide functional evidence that ATRA-mediated upregulation of HLA class II, CD40, and CD86 molecules also translates into acquisition of APC function, experiments were conducted to determine whether ATRA-treated ImC acquired the capability of priming an HLA class II-restricted T-cell response. ImC were cultured for 4 days with increasing concentrations of ATRA and used as stimulators in mixed lymphocyte reactions. In these experiments, allogeneic CD4⁺ T cells (isolated by magnetic bead separation) were used as responders. ATRA treatment resulted in a dose-dependent acquisition of immunostimulatory function. Maximal stimulation of T-cell proliferation could consistently be achieved using ATRA concentrations of 1 μM, while lower or higher concentrations resulted in significantly inferior allo-stimulatory capacity or significant cell death, respectively.

These studies show that in vitro culture of ImC with ATRA results in the upregulation of the cell surface markers HLA-DR, CD40, and CD86 and in acquisition of APC function in a dose-dependent manner. Maximal stimulation of T-cell proliferation could consistently be achieved using ATRA concentrations of 1 μM, while lower and higher concentrations resulted in significantly inferior allo-stimulatory capacity.

ImC-Mediated Immunosuppression can be Abrogated by ATRA

The objective of these studies was to study the immunosuppressive function of CD33⁺/HLA-DR⁻ImC and to explore the underlying mechanisms of ImC-mediated immunosuppression. CTL analysis was performed as a functional readout by using MART-1-specific CTL (derived from a MART-1 specific CTL clone) as effectors. MART-1 peptide-loaded T2 target cells were cultured for 5 h with ⁵¹Cr at a 1:1 ratio. Identical numbers of MART-1 peptide-, or control peptide-loaded ImC, isolated from a HLA-A201⁺ RCC patient, were then added to individual effector/target cell aliquots. As controls, peptide-loaded T2 cells (Cold Target), or ImC isolated from the PBMC of a HLA-A2⁺ healthy donor (Donor) were added to individual cultures.

MART-1 peptide-loaded CD33⁺/HLA-DR⁻ ImC, isolated from a RCC patient, significantly inhibited lysis by MART-1-specific CTL, while healthy donor-derived ImC exhibited only modest T-cell suppressive function (Donor ImC). Control targets in the form of MART-1 peptide pulsed T2 cells were consistently lysed. The addition of ATRA [1 μM] resulted in significant abrogation of ImC-mediated immunosuppressive function.

In summary, these experiments indicate that ImC with suppressive activity can be isolated via simplified isolation techniques using the myeloid marker CD33⁺. CD33⁺ ImC significantly inhibited antigen-specific CTL responses in vitro, but their immunosuppressive action could be abrogated by ATRA-mediated differentiation.

Immunosuppressive Action of ImC by Reactive Oxygen Species (ROS).

Impact of ROS: Studies were conducted to investigate whether ImC-induced suppressive activity can be abrogated by inhibitors of superoxide anions, namely superoxide dismutase (SOD) and peroxide (Catalase), as well as by the peroxynitrite scavenger uric acid. For functional analysis, Interferon-γ ELISPOT assays were performed using an identical experimental setup as described (Effectors: MART-1-specific CTL; Targets: MART-1 peptide-loaded T2 cells; RCC and donor-derived ImC). The addition of RCC patient-derived CD33⁺ ImC to the ELISPOT reaction significantly suppressed the numbers of interferon-γ secreting effector cells (ImC). The addition of catalase [500 U/ml] significantly reduced ImC-mediated T-cell suppression, suggesting that H₂O₂ contributes to ImC-mediated T-cell inhibitory function. SOD alone did not significantly reverse ImC-mediated immunosuppression, however the combination of catalase and SOD (SOD converts superoxide anions into peroxide) reversed immunosuppression in a synergistic fashion. Finally, the addition of uric acid to cultures only modestly enhanced immunostimulatory function, suggesting that, in this experimental system, peroxynitrite was not a major contributor to ROS-mediated immunosuppression. No significant production of ROS was observed by ImC isolated from a healthy volunteer (Donor).

Impact of Nitric Oxide: In separate experiments, analyses were carried out to determine the impact of ImC-derived NO on suppression of T-cell function in vitro. Since inhibition of iNOS (inducible nitroxide synthetase) using the competitive inhibitor NMMA (ω-N-methyl-L-arginine) and of endogenous NO radicals with carboxy-PTIO greatly interfered with IFN-γ production of T cells, ELISPOT assays could not be used as a functional readout. Therefore, MART-1 peptide pulsed ImC was incubated with a MART-1 peptide specific CTL clone and these cells were subsequently stained with the fluorogenic probe, DAF-FM diacetate (4-amino-5-methylamino-2′,7′-difluorescein, a cell permeable molecule that forms a fluorescent benzotriazole after reaction with endogenous NO). ImC isolated from a RCC patient constitutively expressed NO and NO production was further enhanced after co-culture with CTL. In contrast, only low levels of NO production could be detected in ImC isolated from a healthy donor after co-culture with MART-1 specific CTL that only insignificantly increased after antigen-specific stimulation. Similar results were obtained when intracellular ROS produced by ImC were analyzed. For detection of ROS produced by ImC, the fluorogenic dichlorodihydrofluorescein diacetate (H₂DCFDA) was used. RCC patient-derived ImC constitutively produced ROS and ROS levels were greatly enhanced after co-culture with CTL, while no significant production of ROS could be observed in ImC isolated from a healthy donor.

In summary, these studies show that ROS and NO are major factors contributing to ImC-mediated T-cell suppression. In RCC patients, ROS and NO production increased significantly after antigen-specific T-cell interaction, while in healthy volunteer-derived ImC, no significant production of ROS or NO could be observed.

Telomerase mRNA-transfected DC Stimulate Antigen-Specific CD4^{+ and CD}8⁺ T-Cell Responses in Patients with Metastatic Prostate Cancer

Telomerase reverse transcriptase (hTERT) represents an attractive target for cancer immunotherapy since hTERT is silent in normal tissues, but reactivated in human tumors, including prostate cancer. hTERT-specific cytotoxic T lymphocytes (CTL) can be stimulated from peripheral mononuclear cells (PBMC) of cancer patients using hTERT mRNA-transfected dendritic cells (DC), and these CTL can recognize and lyse autologous tumor cells. A phase I/II clinical trial has been initiated in which hTERT mRNA-transfected mature DC were administered to 20 patients with metastatic prostate cancer. Among those, 8 subjects received DC transfected with mRNA encoding a chimeric LAMP hTERT protein allowing for concomitant induction of hTERT-specific CD8⁺ and CD4⁺ T-cell responses. Study endpoints were vaccine safety, immunologic, and therapeutic efficacy. Treatment was well tolerated in all subjects, and no major toxicities or adverse events were encountered. Intense infiltrates of hTERT-specific CD4⁺ and CD8⁺ T cells were noted at the cutaneous injection sites after repeated vaccination. In 19 of 20 subjects, expansion of hTERT-specific CD8⁺ T cells in the peripheral blood of study subjects was measured, with 0.9% to 1.8% of CD8⁺ T cells exhibiting antigen specificity after 6 weekly injections. Patients immunized with the chimeric LAMP hTERT vaccine developed higher frequencies of hTERT-specific CD4⁺ T cells than subjects receiving DC transfected with the unmodified hTERT template. Moreover, the LAMP-driven enhancement of CD4 immunity resulted in improved CTL-mediated killing of hTERT-expressing targets, indicating that an improved CD4⁺ T-cell response can augment a CTL response. Vaccination was further associated with a reduction of PSA velocity (doubling time) and molecular clearance of circulating micrometastases.

In summary, these data demonstrate that repeated administration of hTERT mRNA-transfected DC is capable of stimulating potent, antigen-specific T-cell responses in metastatic prostate cancer patients. Vaccination with LAMP hTERT mRNA-transfected DC leads to enhanced stimulation of hTERT-specific CD4⁺ T cells in vivo.

Isolation and Phenotypic Characterization of ImC from Peripheral Blood.

Peripheral blood cells from subjects will be separated from mononuclear cells by Histopaque gradient centrifugation. The mononuclear cells will be depleted of CD3⁺ cells by using magnetic beads and will be re-suspended in RPMI 1640 medium containing 10% fetal bovine serum, HEPES buffer, penicillin/streptomycin and 30 ng/ml of GM-CSF to sustain cell viability. After a 48-hour incubation step, monocytic cells will be isolated using an OptiPrep™ density gradient. Monocytic cells will be labeled with PE-conjugated lineage-specific (CD3, CD14, CD19, CD56) antibodies and FITC-conjugated HLA-DR antibodies. ImC subsets will be isolated by FACS sorting Lin⁻/HLA-DR⁻ cells.

ImC populations isolated using the myeloid marker CD33 will be further tested and evaluated. ImC preparations will be tested phenotypically and functionally, by extensive FACS staining for HLA class I, class II, CD13, CD15, CD16, CD18, CD33, CD11b, and c.

Demonstration of ImC-Mediated Suppressive Function.

The ability of ImC to suppress stimulation of allogeneic T cells by autologous DC in vitro will be initially tested in allogeneic mixed lymphocyte reactions (MLR) and ELISPOT assays, allowing functional analysis in a setting of limited cell availability.

Mixed lymphocyte reaction In brief, 5×10⁵ allogeneic T cells and 1×10⁴ autologous DC will be plated in each well of 96-well round-flat bottom plates. After 24 hours, increasing numbers of autologous ImC will be added to the MLR reaction. After 5 additional days of culture, 1 μCi [³H]thymidine will be added to each well and incubated for 18 h. Tritiated thymidine uptake will be analyzed by liquid scintillation counting.

ELISPOT analysis: ImC-mediated impact on human CD4⁺ or CD8⁺ T-cell subsets will be analyzed by Interferon-γ ELISPOT analysis. For example, the ability of ImC to suppress CD8⁺ T-cell responses can be examined using Flu peptide-specific CTL, generated from the peripheral blood of a HLA-A2⁺ donor. Monocyte-derived DC and Lin⁻/HLA-DR⁺ cells (DC-enriched fraction), a by-product after cell sorting, will be pulsed with influenza peptide (10 mM), washed, and incubated in complete RPMI 1640 medium with T cells in 24-well plates in the presence of IL-2. T cells will be restimulated with peptide-pulsed DC on days 7 and (if necessary) on day 14. IL-2 will be added immediately after restimulation. CTL will be harvested on day 7 and 14 and used for ELISPOT analysis. After blocking wells with complete medium, 1×10⁵ T cells and 1×10⁴ peptide-pulsed DC will be added to each well of 96-well nitrocellulose plates (Multiscreen-IP, Millipore, Bedford, Mass.) precoated with 2 μg/ml Interferon-γ capture antibody (Endogen, Rockford, Ill.) according to the manufacturer's recommendations (BD Biosciences Pharmingen, San Diego, Calif.). Plates will be incubated for 20 hours at 37° C., and biotinylated Interferon-γ detection antibody (Endogen, Rockford, Ill.) will be added to each well. Cells will be incubated for an additional two hours at room temperature, then with streptavidin-alkaline phosphatase (1 μg/ml; Sigma, St. Louis, Mo.) and plates will be developed with substrate (Kirkegaard & Perry Laboratories, Gaithersburg, Md.). After washing, spots will be counted using an automated Zeiss KS Elispot Compact reader (Carl Zeiss Inc., Minneapolis, Minn.).

Alternative analyses: ImC function can also be analyzed by other complementary assays. For example, the impact of ImC on CD4⁺ T-cell immunity can be evaluated by using proliferation and flow-cytometry-based analyses.

Analysis to Determine the Presence of Mediators Supporting ImC Development and Function.

In order to characterize the cytokine environment in the peripheral blood of cancer patients, expression of cytokines in the sera of patients will be compared to healthy donor sera for the presence of VEGF, GM-CSF, M-CSF, IL-6, IL-10, and IL-13 through the use of ELISA assays (R&D Systems). To analyze the renal tumor milieu for expression of cytokines involved in the development, survival and suppressive function of ImC, mRNA copy numbers of IL-3, IL-6, IL-10, IL-13, TGF-β, VEGF, M-CSF, G-CSF, and GM-CSF will be quantitatively analyzed by real-time PCR from both healthy and cancerous tissues (harvested during nephrectomy). RNA will be extracted from homogenized freshly isolated tissue by use of an RNA isolation kit (Qiagen). Isolated RNA will be reverse transcribed into cDNA using Superscript II reverse transcriptase and random hexamer primers. mRNA copy numbers will be determined by amplification with sequence-specific primer pairs and analyzed by SYBR green-based real-time PCR. In preliminary studies, serum levels of the cytokines IL-6, IL-10, IL-13, VEGR, M-CSF, and PGE₂ were measured from a healthy donor and a RCC patient with metastatic disease. Levels of cytokine expression implicated in ImC development were consistently elevated in the cancer patient when compared to the healthy donor control.

Analysis to Determine the Presence of Mediators and By-Products of Oxidative Stress.

In order to determine ROS activity in RCC patient sera, the levels of the peroxidized lipid by-products malondialdehyde and 8-iso-Prostaglandin F2α will be detected by thiobarbituric acid conversion of malondialdehyde into urea for spectrophotometric evaluation and by detection of 8-iso-Prostaglandin F2α (Stressgen). In order to monitor tumor-mediated expression of ROS-producing enzymes messenger RNA purified from freshly isolated normal and cancerous tissue will be evaluated for the presence of myeloperoxidase, iNOS, and arginase I transcripts.

Induction of ImC Differentiation by using Growth Factors or Differentiation Agents, Such as All-Trans Retinoic Acid (ATRA).

In these studies, the differentiating properties of ATRA and other differentiation agents (Fenretinide and 9-cis-retinoic acid) will be evaluated for human application in vaccination settings. Several experimental conditions will be tested to define optimal dosing and treatment schedules to facilitate ImC differentiation. In vitro cultures of monocytic fractions containing ImC will be exposed to increasing ATRA concentrations (range 1 nM-1 μM) and cultures will be assessed for the presence of ImC after one week of treatment. In addition, differentiation will be monitored by measuring immunostimulatory function and HLA-class II acquisition. Once an optimal dose range is determined, ATRA will be added to cultures at multiple time points, such as day 0 only, day 0 and day 3 only, or day 0, day 2, and day 4.

In alternative methods, healthy donor monocytes can be used as ImC surrogates and their differentiation into immature DC can be monitored. In this procedure, monocytes will be cultured in GM-CSF-containing medium and increasing doses of ATRA will be added. Following a 5 day culture period, cells will be characterized for the presence or absence of DC.

Clinical protocol: treatment and allocation of patients. Between 12 and 18 patients with metastatic RCC after nephrectomy will be enrolled in a study to assess the safety, ImC frequency and immunologic response, and to monitor eventual clinical responses to therapy with ATRA (Tretinoin), followed by vaccination with LAMP hTERT mRNA-transfected DC. All patients must have confirmed metastatic RCC and will be screened to ascertain that they meet the eligibility criteria.

Following nephrectomy, eligible subjects will undergo leukapheresis to generate the vaccine. PBMC will be cultured with GM-CSF and IL-4 to produce DC. Immature DC will be transfected with LAMP hTERT mRNA via electroporation, followed by maturation using the proinflammatory cytokines TNF-α, IL-1β, IL-6, and PGE₂. DC will be cryopreserved until administration. Frozen aliquots will be tested for sterility (negative bacterial, fungal, and mycoplasma) and endotoxin (<5 EU/kg body weight per injection dose) prior to administration. Prior to vaccination, subjects will receive ATRA (Tretinoin) capsules with written and verbal instructions. All cohorts will receive 45 mg/m² per day (divided into two oral doses, given BID). Cohort one will receive ATRA (Tretinoin) capsules for seven (7) days, cohort two for 14 days, and finally, cohort three for twenty-eight (28) days followed by vaccination with LAMP hTERT mRNA-transfected DC. DC vaccinations will be given on a weekly basis for a total of 6 vaccinations, consisting of 1×10⁷ LAMP hTERT mRNA-transfected DC. Subjects will be monitored for safety, immunologic, and clinical responses. Furthermore, ImC will be tracked and enumerated from the peripheral blood of all study subjects. Patients will be followed for one year or until they are withdrawn from study or decide to undergo alternative treatment.

Immunological Monitoring Interferon-γ ELISPOT assays will be used to detect vaccine-induced hTERT-specific CD8⁺ and CD4⁺ T-cell responses from vaccinated subjects. If a significant increase in Interferon-γ-expressing cells is observed (>2-fold increase compared to pre-vaccination baseline), other complementary immunological assays (CTL, proliferation, and flow cytometry-based analyses) will be performed on immunological responders.

ELISPOT analysis of hTERT-specific T lymphocytes PBMC samples obtained during the course of vaccination will be analyzed without restimulations. PBMC will be thawed and reconstituted according to standard operating procedures and stimulated for 18 hours with hTERT mRNA-transfected DC on microwell plates coated with Interferon-γ, IL-2 (Th-1 cytokines), or IL-5 (Th-2 cytokine) capture antibody. PBMC will be exposed to other antigenic stimuli in the form of GFP-mRNA (control), hTERT protein loaded, or hTERT mRNA-transfected DC. As a background control, cells will be also tested for spontaneous cytokine secretion. Spot forming cells will be counted using a fully automated ELISPOT reader (Zeiss, Thornwood, N.Y.).

Analysis of hTERT-specific cytotoxicity Standard ⁵¹Cr cytotoxicity assays will be used for CTL analysis. PBMC from vaccinated patients be analyzed for their capability to lyse their cognate target cells. Possible target cells will include a) hTERT mRNA-transfected DC, b) autologous BLCL, and c) HLA-matched allogeneic tumor cells. As control targets, DC transfected with GFP mRNA, K562 cells (to exclude NK-mediated lysis) and Daudi cells (to account for LAK activity) will be used.

Multiparameter flow cytometry. Experiments will be conducted to directly test and compare multiparameter flow cytometry data from fresh or cryopreserved PBMC samples following 6 hour stimulation with hTERT mRNA or hTERT protein-loaded DC. As controls, DC transfected with GFP or PSA mRNA will be used. Multiparameter flow cytometry results from pre- and post-immunization samples will be compared with gating on CD4⁺ and CD8⁺ T cells and an aim to quantitatively analyze cells expressing Th-1 or Th-2 cytokines such as Interferon-γ, TNF-α, IL-2, IL-13, IL-4, and IL-5 within these T-cell populations.

Analysis of hTERT-Specific CD4⁺ T Lymphocytes

The following T-helper cells assays will be conducted: a) Multiparameter flow cytometry for detection of intracellular cytokine producing T cells, b) Standard proliferation assays (based on [³H]thymidine incorporation), and c) ELISA-based detection of T-helper cytokine expression. To determine hTERT-specific CD4⁺ proliferation, autologous DC transfected with hTERT, LAMP hTERT mRNA, or GFP RNA (which is used as a control antigen) will be used as stimulators. Cryopreserved, RNA-transfected DC are thawed and co-cultured cultured with autologous PBMC at various responder:stimulator ratios. CD4⁺ T cells isolated by magnetic bead separation will be incubated for 3 days. After 4 days of culture, 1 μCi of methyl-[³H]-thymidine (NEN™, Boston, Mass.) will be added to each well and incubation will be continued for an additional 18 hours. Cells will be collected onto Glass fiber filters (Wallac, Turku, Finland) with a cell harvester and uptake of thymidine will be determined using a liquid scintillation counter.

To analyze T-helper cytokine expression and to further characterize the nature of the responding T-helper cells (e.g. Th1 versus Th2 type T cell responses), cytokines secreted into the supernatant by cultured responding T cells will be analyzed by ELISA. Supernatants from the cultures will be analyzed for the presence of Interferon-γ (Th1 marker) as well as for IL-5, IL-13, and IL-4 secretion (Th2 markers). For cytokine flow cytometry (CFC), whole blood obtained either prior to or after vaccine therapy will be incubated overnight with autologous hTERT mRNA or control RNA-transfected DC in the presence of co-stimulatory antibodies to CD28. The blood samples will then be treated with EDTA, erythrocytes will be lysed and leukocytes fixed, permeabilized, and stained for intracellular cytokines (TNF-α, Interferon-γ, CD4, and CD69). Cells will be analyzed by flow cytometry and cytokine⁺/CD69⁺ cells will be enumerated as a percentage of the total CD4⁺ T cell number.

Although the present process has been described with reference to specific details of certain embodiments and examples thereof, it is not intended that such details should be regarded as limitations upon the scope of the invention except as and to the extent that they are included in the accompanying claims.

Throughout this application, various patents, patent publications and non-patent publications are referenced. The disclosures of these patents, patent publications and publications in their entireties are incorporated by reference into this application in order to more fully describe the state of the art to which this invention pertains.

### REFERENCES

1. Heiser, A. et al. Dendritic cells transfected with prostate specific antigen RNA stimulate PSA specific T cell responses in metastatic prostate cancers. J. Clin. Invest. 158, 820-829 (2002).
2. Su, Z. et al. Telomerase mRNA-transfected dendritic cells stimulate antigen-specific CD8+ and CD4+ T cell responses in patients with metastatic prostate cancer. J. Immunol. 174, 3798-807 (2005).
6. Curiel, T. J. et al. Specific recruitment of regulatory T cells in ovarian carcinoma fosters immune privilege and predicts reduced survival. Nat. Med. 10, 942-9 (2004).
10. Olsen, E. et al. Pivotal phase III trial of two dose levels of denileukin diftitox for the treatment of cutaneous T-cell lymphoma. J. Clin. Oncol. 19, 376-88 (2001).
13. Wing, K., Ekmark, A., Karlsson, H., Rudin, A. & Suri-Payer, E. Characterization of human CD25+CD4⁺ T cells in thymus, cord and adult blood. Immunology 106, 190-9 (2002).
14. Morgan, M. E. et al. Expression of FOXP3 mRNA is not confined to CD4+CD25+ T regulatory cells in humans. Hum. Immunol. 66, 13-20 (2005).
15. Woo, E. Y. et al. Regulatory CD4(+)CD25(+) T cells in tumors from patients with early-stage non-small cell lung cancer and late-stage ovarian cancer. Cancer Res. 61, 4766-72 (2001).
16. Rosolen, A. et al. Expression of interleukin-2 receptor beta subunit in hematopoietic malignancies. Blood 73, 1968-72 (1989).
18. Heiser, A. et al. Human dendritic cells transfected with RNA encoding prostate specific antigen stimulate prostate specific CTL responses in vitro. J. Immunol. 164, 5508-5514 (2000).
19. Heiser, A. et al. Human Dendritic Cells Transfected with Renal Tumor RNA Stimulate Polyclonal T-Cell Responses against Antigens Expressed by Primary and Metastatic Tumors. Cancer Res. 61, 3388-93 (2001).
20. Heiser, A. et al. Induction of polyclonal prostate cancer specific cytotoxic T lymphocytes using dendritic cells transfected with amplified tumor RNA. J. Immunol. 166, 2953-2960 (2001).
21. Baecher-Allan, C., Viglietta, V. & Hafler, D. A. Inhibition of human CD4(+)CD25(+high) regulatory T cell function. J. Immunol. 169, 6210-7 (2002).
22. Frankel, A. E., Powell, B. L. & Lilly, M. B. Diphtheria toxin conjugate therapy of cancer. Cancer Chemother. Biol. Response Modif. 20, 301-13 (2002).
23. Goldrath, A. W., Luckey, C. J., Park, R., Benoist, C. & Mathis, D. The molecular program induced in T cells undergoing homeostatic proliferation. Proc. Natl. Acad. Sci. USA 101, 16885-90 (2004).
24. Su, Z. et al. Immunological and Clinical Responses in Metastatic Renal Cancer Patients Vaccinated with Tumor RNA-transfected Dendritic Cells. Cancer Res. 63, 2127-2133 (2003).

25. Tsaknaridis, L. et al. Functional assay for human CD4+CD25+ Treg cells reveals an age-dependent loss of suppressive activity. J. Neurosci. Res. 74, 296-308 (2003).

TABLE 1
Patient Characteristics and Treatment Assignments

| | | | | | | | | | | | Time intervals^{b} | | | |
|---|---|---|---|---|---|---|---|---|---|---|---|---|---|---|
| | | | | | | | | | | | (in months) | | | |
| | | | | | Meta- | Treat- | | | | | | | Last | |
| | | | | | stases | ments | DAB ₃₈₉ | Treg | | Dip- | | | F/U | |
| Subject | | | KPS | Diag- | (Study | Prior | IL-2 Dose | Depletion | | theria | Dx Met | Nx to | after | |
| ID | Age | Sex^{a} | (%) | nosis | Entry) | to Vac | (μg) | (%) | DC Dose | Titer | to Vac | Vac | Vac | Status |
| JCB-01-DAB | 58 | M | 80 | RCC | LN/BN | SRG | 2412 | 74 | 2 × 10⁷ | 1:6561 | 36 | 36 | 15 | AWD |
| HM-02-DAB | 54 | M | 90 | RCC | BN | SRG/Imm/Ch/ | 1494 | 88 | 3 × 10⁷ | 1:243 | 39 | 38 | 14 | AWD |
| | | | | | | XRT | | | | | | | | |
| JVG-03-DAB | 56 | F | 90 | OVA | ST | SRG/Ch/Imm | 900 | 37 | 3 × 10⁷ | 1:19683 | 35 | N/A | 8 | AWD |
| HMT-04-DAB | 69 | F | 90 | RCC | PN | SRG | 1590 | 77 | 3 × 10⁷ | negative | 2 | 2 | 2 | DOD |
| WDH-01-RCC | 60 | M | 70 | RCC | LN/BN | SRG/Imm | N/A | N/A | 3 × 10⁷ | N/D | 10 | 10 | 9 | DOD |
| JDG-02-RCC | 61 | M | 75 | RCC | PN | SRG | N/A | N/A | 3 × 10⁷ | N/D | 6 | 6 | 4 | DOD |
| JWV-03-RCC | 67 | M | 85 | RCC | PN/ST | SRG/Imm | N/A | N/A | 3 × 10⁷ | N/D | 16 | 16 | 3 | AWD |
| M-K-04-RCC | 67 | M | 95 | RCC | PN | SRG/Imm | N/A | N/A | 3 × 10⁷ | N/D | 10 | 9 | 1 | AWD |

^{a}M, male; F, female; KPS, Karnofsky Performance Status; RCC, renal cell carcinoma; OVA, ovarian cancer; BN, bony metastases; LN, lymph node metastases; PN, pulmonary nodule metastases; ST, soft tissue; SRG, surgery; Ch, chemotherapy; XRT, radiation therapy; Imm, immunotherapy; Treg, regulatory T cells; DC, dendritic cell; N/A, not applicable; N/D, not determined; AWD, alive with disease; DOD, dead of disease.
^{b}Time intervals: Dx Met to Vac, time between first diagnosis of metastatic disease and first DC vaccination; Nx (nephrectomy) to Vac, time between nephrectomy and first DC vaccination; Last F/U (follow-up) after Vac, time interval between first vaccination and last clinical/radiological follow-up.

TABLE 2
Patient Characteristics and Treatment Assignments
Vaccine: Tummor RNA-transfected DC

| | | | | | | | | | | | | | Time | | | |
|---|---|---|---|---|---|---|---|---|---|---|---|---|---|---|---|---|
| | | | | | | | | | | ELISPOT | | | Intervalsⁱ | | | |
| | | | | | | | | | | (Fold | | | (months) | | | |
| | | | | | Meta- | Treat- | | # of | Treg | increase | | | Dx | | | |
| | | | | | stases^{e} | ments | DAB | Treg | Deple- | after | | Dip- | Met | Nx | F/U | |
| Subject | | | KPS^{c} | Diag- | (Study | Prior | Dose^{g} | (% | tion^{h} | vaccination) | | theria | to | to | after | Sta- |
| ID^{a} | Age | Sex^{b} | (%) | nosis^{d} | Entry) | to Vac^{f} | (μg) | CD4⁺) | (%) | CD8⁺ | CD4⁺ | Titer | Vac | Vac | Vac | tus^{j} |
| 01-RCC-DAB | 58 | M | 80 | RCC | LN/BN | Nx/MR | 2412 | 4.6 | 74 | 16.0 | 7.6 | 1:6561 | 36 | 36 | 24 | AWD |
| 02-RCC-DAB | 54 | M | 90 | RCC | BN | Nx/MR/ | 1494 | 2.5 | 72 | 12.5 | 6.3 | 1:243 | 39 | 39 | 21 | AWD |
| | | | | | | Cyt/Ch/ | | | | | | | | | | |
| | | | | | | XRT | | | | | | | | | | |
| 03-RCC-DAB | 69 | F | 90 | RCC | PN | Nx/MR | 1590 | 3.7 | 68 | 5.4 | 2.2 | negative | 3 | 2 | 1 | DOD |
| 04-RCC-DAB | 60 | M | 90 | RCC | PN | Nx/Cyt/ | 1906 | 3.8 | 76 | 7.4 | 9.7 | negative | 170 | 204 | 5 | AWD |
| | | | | | | Ch/MR | | | | | | | | | | |
| 05-RCC-DAB | 57 | M | 100 | RCC | ST/PN | Nx/MR | 1566 | 2.8 | 57 | 7.8 | 31.0 | N/D | 8 | 155 | 5 | AWD |
| 06-RCC-DAB | 61 | M | 90 | RCC | BN | Nx/XRT/ | 1301 | 4.2 | 26 | 8.0 | 6.8 | negative | 14 | 26 | 6 | AWD |
| | | | | | | Cyt/MR | | | | | | | | | | |
| 07-OVA-DAB^{k} | 56 | F | 90 | OVA | ST | TAH/Ch/ | 900 | 3.7 | 35 | 7.8 | 4.5 | 1:19683 | 35 | N/A | 16 | AWD |
| | | | | | | MR/Cyt | | | | | | | | | | |
| 08-RCC | 60 | M | 70 | RCC | LN/BN | Nx/MR/ | N/A | 2.7 | N/A | 0.3 | 1.0 | N/D | 10 | 10 | 8 | DOD |
| | | | | | | Cyt | | | | | | | | | | |
| 09-RCC | 61 | M | 75 | RCC | PN | Nx/MR | N/A | 3.2 | N/A | 5.1 | 2.0 | N/D | 5 | 5 | 3 | DOD |
| 10-RCC | 67 | M | 85 | RCC | PN/ST | Nx/Cyt/ | N/A | 3.0 | N/A | 3.2 | 4.5 | N/D | 16 | 16 | 11 | AWD |
| | | | | | | MR | | | | | | | | | | |
| 11-RCC | 67 | M | 95 | RCC | PN | Nx/Cyt/ | N/A | 4.4 | N/A | 2.1 | 2.0 | N/D | 8 | 9 | 9 | AWD |
| | | | | | | MR | | | | | | | | | | |

^{a}Subject ID: subject identification; DAB, pretreatment with DAB₃₈₉IL-2 4 days prior to DC vaccination; RCC, metastatic renal cell carcinoma; OVA, metastatic ovarian carcinoma.
^{b}Sex: M, male; F, female.
^{c}KPS: Karnofsky Performance Status.
^{d}Diagnosis: RCC, renal cell carcinoma; OVA, ovarian cancer; PCA, prostate cancer.
^{e}Metastases: LN, lymph node; BN, bony; PN, pulmonary nodule; ST, soft tissue; PSA, hormone refractory with rising PSA despite continued androgen ablation.
^{f}Treatment Prior to Vaccination: Nx, nephrectomy; MR, metastatic tumor resection; Cyt, cytokines; Ch, chemotherapy (5-FU); XRT, radiation therapy; TAH, total abdominal hysterectomy; RP, radical prostatectomy; H, hormones.
^{g}DAB ₃₈₉ IL-2 Dose: 18 μg/kg.
^{h}Treg: regulatory T cells.
ⁱTime Intervals: Dx Met to Vac, time between first diagnosis of metastatic disease and first DC vaccination; Nx to Vac, Time between nephrectomy first DC vaccination; F/U (follow-up) after Vac, time interval between first vaccination and last clinical/radiological follow-up.
^{j}Status: AWD, alive with disease; DOD, dead of disease.
^{k}Subject with ovarian carcinoma, not included into statistical analysis
DC, dendritic cells;
N/A, not applicable;
N/D, not determined.

What is claimed is:

1. A method of enhancing an immune response in a subject, comprising administering to the subject a reagent that targets a cell having immunosuppressive activity, in an amount effective in reducing the immunosuppressive activity of the cell, thereby enhancing an immune response in the subject.

2. A method of treating cancer in a subject, comprising:
a) first administering to the subject a reagent that targets a cell having immunosuppressive activity in an amount effective in reducing the immunosuppressive activity of the cell; and
b) subsequently administering to the subject a reagent that targets the cancer in the subject and/or elicits an immune response to the cancer cells of the subject.

3. A method of treating an infection in a subject, comprising:
a) first administering to the subject a reagent that targets a cell having immunosuppressive activity in an amount effective in reducing the immunosuppressive activity of the cell; and
b) subsequently administering to the subject a reagent that targets an agent that is causing an infection in the subject.

4. The method of claim 1, wherein the reagent is selected from the group consisting of an antibody, a ligand, an immunotoxin and a fusion protein comprising a targeting moiety and a toxic moiety.

5. The method of claim 4, wherein the antibody is selected from the group consisting of an antibody that binds CD25, an antibody that binds CTLA4, an antibody that binds GITR and an antibody that binds FOXP3.

6. The method of claim 4, wherein the fusion protein comprises a catalytic domain of diphtheria toxin and a binding domain of interleukin-2.

7. The method of claim 1, wherein the cell is a regulatory T cell.

8. The method of claim 1, wherein the cell is an immature myeloid cell.

9. The method of claim 2, wherein the cancer is selected from the group consisting of: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, lymphoma, multiple myeloma, Waldenstroom's macroglobulinemia, and heavy chain disease.

10. The method of claim 3, wherein the agent that causes an infection is selected from the group consisting of viral pathogens (e.g., hepatitis type A, hepatitis type B, hepatitis type C, influenza, varicella, adenovirus, herpesvirus, rhinovirus, echovirus, rotavirus, lentivirus, retrovirus, respiratory syncytial virus (RSV), papilloma virus, papova virus, cytomegalovirus, coronavirus, arbovirus, hantavirus, coxsackie virus, mumps virus, measles virus, rubella virus, polio virus, human immunodeficiency virus type I (HIV-I), and human immunodeficiency virus type II (HIV-II); prokaryotic pathogens (e.g., mycobacteria, rickettsia, Mycoplasma spp., Neisseria spp. and Legionella spp., chlamydia; and protozoal pathogens (e.g., Leishmania spp. and Trypanosoma spp).

11. The method of claim 2, wherein the reagent of step (a) is administered at least one day before the reagent of step (b) is administered.

12. The method of claim 2, wherein the reagent of step (a) is administered at least two days before the reagent of step (b) is administered.

13. The method of claim 2, wherein the reagent of step (a) is administered at least three days before the reagent of step (b) is administered.

14. The method of claim 2, wherein the reagent of step (a) is administered at least four days before the reagent of step (b) is administered.

15. The method of claim 2, wherein the reagent of step (a) is administered at least five days before the reagent of step (b) is administered.

16. The method of claim 4, wherein the amount of fusion protein administered is in a range of about 8 μg/kg to about 20 μg/kg.

17. The method of claim 2, wherein the reagent of step (b) is a dendritic cell loaded with messenger RNA encoding a tumor antigen specific for the cancer of the subject.

18. The method of claim 17, wherein the amount of dendritic cells administered is in a range of about 3×10⁷ cells to about 10×10⁷ cells.

19. The method of claim 2, wherein the reagent of step (a) is administered to the subject prior to and not during the administration of the reagent of step (b).

20. The method of claim 3, wherein the reagent of step (a) is administered to the subject prior to and not during the administration of the reagent of step (b).
